(19) United States
(12) Patent Application Publication
(10) Pub. No.: US 2004/0203115 A1
(43) Pub. Date: Oct. 14, 2004

(54) Methods and compositions for production and purification of recombinant staphylococcal enterotoxin b (rseb)

(76) Inventors: Steven L Giardina, Carroll Valley, PA (US); Jianwei Zhu, Frederick, MD (US)

Correspondence Address: Brian W Poor, Townsend and Townsend and Crew, 8th Floor, Two Embarcadero Center, San Francisco, CA, 94111, US

(21) Appl. No.: 10/492,105

(22) PCT Filed: Sep. 27, 2002
(86) PCT No.: PCT/US02/31114

Related U.S. Application Data
(60) Provisional application No. 60/328,017, filed on Oct. 9, 2001.

(51) Int. Cl. C07K 14/195; C08B 37/00

(52) U.S. Cl. 435/69.3; 530/412; 536/53

(57) ABSTRACT

The invention provides processes and compositions for fermentation, recovery and purification of recombinant bacterial superantigens (rSAgs), exemplified by a recombinant staphylococcal enterotoxin B SEB protein mutated for use in administration to mammalian recipient. This process generates an economically viable quantity of rSEB vaccine protein meeting FDA parenteral drug specifications. The purification methods generally involve multiple steps including hydrophobic interaction chromatography (HIC), buffer exchange (desalting), and cation exchange. The final product of the purification is a highly purified rSAg composition satisfying clinical safety criteria and is immunogenic and protective against lethal aerosol challenge in a murine model. The methods and compositions of the invention provide useful tools for treatment of disease and other conditions caused by bacterial SAgs, including food poisoning, bacterial arthritis and other autoimmune disorders, toxic shock syndrome, and insults attributed to the potential use of SAg biowarfare agents.

### BACKGROUND OF THE INVENTION

The pyrogenic exotoxins of Group A streptococci and the enterotoxins of Staphylococcus aureus, also pyrogenic exotoxins, constitute a family of structurally related toxins which share similar biological activities (Hynes et al., Infect. Immun. 55:837-840, 1987; Johnson et al., Mol. Gen. Genet. 203:354-356, 1986, each incorporated herein by reference). The staphylococcal and streptococcal pyrogenic exotoxins also share significant amino acid homology throughout their sequences (Hynes et al., supra; Marrack et al., Science 248:705-7, 1990; Hoffman et al., Infect. Immun. 62:3396-3407, 1994, each incorporated herein by reference). This pyrogenic exotoxin family contains nine main toxin types, and several allelic variants (subtypes) have been described. The gene sequences and deduced amino acid sequences of at least six staphylococcal enterotoxins (“SE”): A, B, C, D, E and H, are known, i.e., SEA, SEB, SEC, SED, SEE, and SEH (Marrack et al., Science 248:705-711, 1990; Reda et al., Infect. Immun. 62:1867-1874, 1994, each incorporated herein by reference). Likewise, the sequences of at least three streptococcal superantigens, SPEA, SPEC, and SSA, are known (Goshorn et al., Infect. Immun. 56:2518-2520, 1988; Reda et al., supra; Weeks et al., Infect. Immun. 52:144-150, 1986, each incorporated herein by reference). Several studies have shown that each of these toxins shares common structural features, for example as demonstrated by immunologic cross reactivity between the toxins (Spero et al., J. Immunol. 120:86-89, 1978; Spero et al., J. Biol. Chem. 253:8787-8791, 1978, each incorporated herein by reference).

The pyrogenic exotoxins of Staphylococcus sp. and Streptococcus sp. share the ability to bind the major histocompatibility complex (MHC) molecules of infected hosts, as well as the variable beta chain of the T-cell receptor complex (TCR), causing an aberrant proliferation of specific T-cell subsets. This property of these toxins is the basis for their common designation as “superantigens” (SAgs), since they do not interact with the MHC and TCR molecules in the manner of conventional antigens.

Important examples of SAgs include exotoxins produced by Staphylococcus aureus and group A streptococci that play a role in bacterial virulence and pathology through their profound effects on the host immune system. There are numerous identified pathological effects described for these and other superantigens, ranging from an acute, but self-limiting, food poisoning from staphylococcal enterotoxins (SEs), to a life-threatening, toxic-shock syndrome that can be caused by most, if not all, bacterial SAgs (Ulrich et al., Vaccine 16:1857-1864, 1998; and Ulrich et al., Trends Microbiol. 3:463-468, 1995, each incorporated herein by reference).

The molecular and physiological events associated with acute SAg toxicity are well documented. Like a number of other virulence factors, SAgs target receptors of the immune system. The MHC class II molecule, HLA-DR (consisting of α and β subunits), serves as the primary cellular receptor (Ulrich, FEMS Immunol. Med. Microbiol. 27:1-7, 2000; and Swaminathan et al., Nature 359:801-806, 1992). When complexed with HLA-DR, the SAg binds to the variable domain of the β subunit (Vβ) of the T-cell antigen receptors (TCRs), primarily by contacts with the complementarity-determining regions 1 and 2 and the hypervariable region 4. Additionally, each SAg acts as a wedge to prevent contacts of antigenic peptides with these combining site elements of the TCR, thus disengaging the normal antigen-specific signal transduction of T cells. However, the response to SAgs involves a substantially greater number of T cells than an antigen-specific response because SAgs bind to multiple subtypes of VP subunits. The excess levels of proinflammatory cytokines released are thought to be responsible for the extreme pathological effects.

Each bacterial superantigen has a distinct affinity to a set of TCR Vβ, and coligation of the MHC class II molecule stimulates T cells in a polyclonal manner (White et al., Cell 56:27-35, 1989; Kappler et al., Science 244:811-813, 1989; and Takimoto et al., Eur. J. Immunol. 140:617-621, 1990, each incorporated herein by reference). Pathologically elevated levels of cytokines that are produced by activated T cells are the probable cause of toxic shock symptoms (Carlson and Sjogren, Cell Immunol 96:175-183, 1985; and Stiles et al., Infect. Immun. 61:5333-5338, 1993, each incorporated herein by reference). In addition, susceptibility to lethal gram-negative endotoxin shock is enhanced by several bacterial superantigens (Stiles et al., Infect. Immun. 61:5333-5338, 1993, incorporated herein by reference).

Staphylococcal enterotoxins (SEs) A through E are the most common cause of food poisoning (Bergdoll, M. S., “Enterotoxins”, in: Staphylococci and Staphylococcal Infections, pp. 559-598, Easom and Aslam, eds., Academic Press, London, 1983, incorporated herein by reference) and are associated with several serious diseases (Schlievert, J. Infect. Dis. 67:997-1002, 1993; and Ulrich et al., Trends Microbiol. 3:463-468, 1995, incorporated herein by reference), such as bacterial arthritis (Schwab et al., J. Immunol. 150:4151-4159, 1993, incorporated herein by reference); and other autoimmune disorders (Posnett, Semin. Immunol. 5:65-72, 1993), and toxic shock syndrome (Schlievert, Lancet 1:1149-1150, 1986; and Bohach et al., Crit. Rev. Microbiol. 17:251-272, 1990, each incorporated herein by reference). The non-enterotoxic staphylococcal super-antigen toxic shock syndrome toxin-1 was first identified as a causative agent of menstrual-associated toxic shock syndrome (Schlievert et al., J. Infect. Dis. 143:509-516, 1981, incorporated herein by reference). Superantigen-producing Staphylococcus aureus strains are also linked to Kawasaki syndrome, an inflammatory disease of children (Leung et al., Lancet 342:1385-1388, 1993, incorporated herein by reference). Although antibodies reactive with superantigen are present at low levels in human sera (Takei et al., J. Clin. Invest. 91:602-607, 1993, incorporated herein by reference), boosting antibody titers by specific immunization is predicted to be efficacious for patients at risk for toxic shock syndrome and the other disorders of common etiology.

Bacterial superantigens cause markedly different clinical syndromes when they are inhaled than are characteristically produced when the antigens are ingested. The effectiveness of SEB as a military biological incapacitating agent, when aerosolized in very low doses, was well documented during the former U.S. offensive biological weapons program. To limit the potential production of material of sufficient quantity to be used as a biological weapon, the Centers for Disease Control and Prevention (CDC) was empowered to regulate nominal quantities of the native molecule as a select agent (Zabriskie, Curr. Opin. Biotechnol. 9:312-318; and Ferguson, JAMA 278:357-360, 1997) A vaccine approach to controlling bacterial superantigen-associated diseases will provide useful tools for preventing or managing such diseases, including in the context of biodefense. It is presumed that specific antibodies will neutralize the pathological effects of SAgs in vaccinated individuals by binding to and sequestering the exotoxin at some stage after entering the body (Bavari et al., J. Infect. Dis. 180:1365-1369, 1999). Inactivation of the SAg is necessary for the purpose of vaccine development, as exposure to the native molecule circumvents critical components of the immune response that are required for antibody production. There are several means of constructing a suitable vaccine candidate (Liljeqvist and Stahl, J. Biotechnol. 73:1-33, 1999). Protein toxins can be treated with a chemical agent, such as formaldehyde, to produce an inactive toxoid vaccine. However, formaldehyde potentially alters the antigenic structure of the protein and is itself toxic (Warren et al., J. Immunol. 111:885-892, 1973; and Wood, et al., FEMS Immunol. Med. Microbiol. 17:1-10, 1997). The process for toxin inactivation is considerably long and poses an undue risk to both the environment and the safety of manufacturing personnel. As a safer alternative to the toxoid approach, vaccines can also be designed wherein the SAg is modified by limited site-directed mutagenesis within conservative receptor-binding motifs. These structurally conserved motifs are common among all bacterial SAgs (Ulrich et al., Vaccine 16:1857-1864, 1998; Bavari et al., J. Infect. Dis. 174:338-345, 1996; and Ulrich et al., Nature Struct. Biol. 2:554-560, 1995) and have been proven useful for designing effective recombinant SAg proteins for vaccine use.

In this context, a three-dimensional structural homology model of SEA complexed with the MHC class II molecule was developed (Ulrich et al., Nature Struct. Biol. 2:554-560, 1995, incorporated herein by reference) from the structural alignment of SEA and unbound SEB molecules (Swaminathan et al., Nature 359:801-806, 1992, incorporated herein by reference) and co-crystallographic data of the complex between SEB and the human MHC class II molecule, HLA-DR1 (Jardetzky et al., Nature 368:711-718, 1994, incorporated herein by reference). MHC class II molecule and TCR-binding modes, confirmed by site-specific mutagenesis, are conserved between SEB and SEA (Ulrich et al., Nature Struct. Biol. 2:554-560, 1995, incorporated herein by reference). The MHC class II-binding surface of SEA and SEB consists of a conserved polar pocket and hydrophobic loop. Mutation of a critical tyrosine (SEA residue 92) within the polar pocket is postulated to disrupt hydrogen bonding to lysine 39 of the DRα subunit and results in a 1000-fold reduction in SEA binding affinity. TCR interactions map to nonconserved residues within a shallow surface cavity of SEA (Ulrich et al., Trends Microbiol. 3:463-468, 1995, incorporated herein by reference) and SEB (Kappler et al., J. Exp. Med. 175:387-396, 1992, incorporated herein by reference). Thus, the mutation tyrosine 64 to alanine, within this site of SEA, results in diminished T cell recognition (Ulrich et al., supra, 1995).

Despite the recognized value of recombinant SAg proteins for vaccine use, there have yet to be developed effective methods and compositions for large scale production of clinical grade, recombinant SAg protein for this purpose. Ulrich et al., (supra, 1995) report partial purification of a recombinant SEA preparation by CM Sepharose chromatography followed by preparative isoelectric focusing, and dialysis. However, this process has not been shown to be effective to yield large quantities of clinical grade SEA free of endotoxin and other contaminants and useful for administration to a mammalian recipient. Accordingly, there is an urgent need in the art for an efficient protocol for selectively isolating bacterial SAgs from crude preparations, particularly crude starting preparations obtained from recombinant expression systems. Such protocols that were heretofore lacking in the art should be applicable to starting material from varying sources, including fermentation broth and lysed bacterial, eukaryotic or mammalian cells, to supply clinical needs.

Surprisingly, the present invention fulfills these needs and satisfies additional objects and advantages that will become apparent from the following description. As such, the present invention makes possible large scale production of recombinant bacterial SAgs, particularly recombinant Staphylococcal enterotoxin B, in a highly purified form and in quantities sufficient for therapeutic uses, such as, for example, biodefense and treatment of sepsis-related conditions, such as toxic, shock, and other diseases and conditions in mammals caused by bacterial SAgs.

### SUMMARY OF THE INVENTION

The instant invention provides robust and reproducible fermentation and purification processes and compositions that enable large-scale manufacturing of recombinant bacterial superantigens (SAgs), as exemplified by the recombinant staphylococcal enterotoxin B (rSEB). In one embodiment of the invention, rSEB is produced under CGMP conditions, and is demonstrated to be stable, immunogenic, and immunogenically effective to protect mammalian subjects against lethal exposure to the native SEB toxin. Briefly, this exemplary embodiment of the invention provides a process for the fermentation, recovery and purification of a rSEB protein. This process generates an economically viable quantity of rSEB protein meeting Food and Drug Administration (FDA) parenteral drug specifications. The final rSEB product of the process is shown to be immunogenic and protective against lethal aerosol challenge in a murine model predictive of immunogenic activity in other mammalian subjects, including human and non-human primates.

In exemplary embodiments of the invention, a method for high yield purification of a recombinant bacterial superantigen (SAg) is provided. The method generally comprises the steps of subjecting a starting load material containing a recombinant SAg and one or more contaminants (e.g., a bacterial endotoxin, lipopolysaccharide, DNA, and the like) to a hydrophobic interaction chromatography (HIC) substrate. From the HIC, a flow through fraction is collected that comprises HIC-purified recombinant SAg partially or completely separated from the endotoxin. The HIC-purified recombinant SAg is subjected to suitable buffer exchange process to desalt the HIC-purified SAg fraction. The resulting product is contacted with a cation exchange matrix under conditions sufficient to bind the recombinant SAg to the cation exchange substrate while not substantially binding contaminants. The recombinant SAg is then eluted from the cation exchange substrate to provide a substantially purified, clinical grade SAg product suitable for use in a mammalian subject.

In alternative embodiments, the starting load material comprises the recombinant SAg solubilized from an ammonium sulfate pellet form obtained from a recombinant cell lysate. This ammonium sulfate precipitate has been found to be suitable for long term storage of recombinant SAg prior to further purification and finishing to form the final product. In more detailed embodiments, the HIC substrate is a resin comprising a propyl, butyl, octyl, or phenyl functional group, for example a low or high substitution phenyl functional group (e.g., depyrogenated phenyl-sepharose). The buffer exchange of HIC-purified recombinant SAg in preparation for cation exchange optionally comprises desalting to separate ammonium sulfate from the HIC-purified recombinant SAg. In other detailed embodiments, the recombinant cell lysate is a lysate of recombinant E. coli cells containing an expression construct that comprises a recombinant bacterial SAg gene operably linked to one or more expression control elements to direct expression of the recombinant SAg protein following induction. In yet other detailed embodiments the rSAg is staphylococcal enterotoxin B (SEB), as exemplified by a rSEB modified by amino acid substitutions at position 89 (from tyrosine to alanine), position 45 (from leucine to arginine), and position 94 (from tyrosine to alanine).

In additional aspects of the invention, a novel bacterial fermentation process for high yield production of a recombinant bacterial superantigen (SAg) is provided. This fermentation method comprises the steps of culturing recombinant E. coli cells from a Master Cell Bank containing an expression construct comprising a recombinant bacterial SAg gene operably linked to one or more expression control elements to direct expression of a recombinant SAg protein in a sterile seed medium to yield a seed culture. Typically this expansion stage is carried out two or more times to form a second and even a third seed culture. When the seed culture is used to form a second seed culture the cells are typically grown to late log phase or early stationary phase (OD₆₀₀ of about 6 to about 10) and are monitored for rate of expansion and other parameters to determine and the overall health and viability of the culture prior to proceeding to the next phase. A further aspect of the present invention is the formulation of a seed and production media that does not comprise animal protein. It has been found that vegetable proteins, including those from soy can be used to replace animal protein as a protein source with a substantial yield of protein product. Further, the seed and culture media can comprise one or more trance elements comprising NH₄SO₄, ZnSO₄.7H₂O, CuSO₄.5H₂, MnSO₄.H₂O, FeCl₃.6H₂O, CoCl₂.6H₂O, or Na₂MoO₄.2H₂O. Also, it has been found that the addition of glucose to the media inhibits the expression of the SAg and therefore glucose has been excluded from the medium.

The recombinant E. coli cells of the final seed culture, typically the first or second seed culture, are used to seed a sterile production medium for the culturing and expansion of the cells to form a production culture. Once the production culture has reached an OD₆₀₀ of about 5 to about 20 (more typically 10 to about 15) the culture is induced to express the recombinant SAg. The recombinant E. Coli cells of the production culture are disrupted to yield a lysate containing the recombinant SAg (rSAg), which is later recovered from the lysate. At least 50-60% of the rSAg is recovered in a soluble form. In certain embodiments, greater than 60% of the recombinant bacterial SAg is recovered in a soluble form, and in other embodiments at least 75-80% of the recombinant bacterial SAg is recovered in a soluble form. In more detailed embodiments, the rSAg is staphylococcal enterotoxin B (SEB), as exemplified by a rSEB modified by amino acid substitutions at position 89 (from tyrosine to alanine), position 45 (from leucine to arginine), and position 94 (from tyrosine to alanine).

The instant invention also relates to the rSAg compositions and formulations prepared by the processes of the invention, and to uses for these compositions and formulations. In particular, the methods and compositions of the invention are useful for producing clinical grade rSAgs as an immunogen or a vaccine agent. In this regard, the methods and compositions herein provide for vaccination against a variety of diseases and conditions associated with SAg exposure. For example, staphylococcal enterotoxins are the most common cause of food poisoning and are associated with several serious diseases, including bacterial arthritis, other autoimmune disorders, and toxic shock syndrome. In addition, SEB is considered a potential biowarfare agent and is thus a threat to both battlefield troops and as a possible terrorist weapon of mass destruction. The production of large quantities of relatively pure, biologically active SAg molecules as immunogens is therefore important for generating effective tools to moderate, treat or prevent these and other diseases and conditions.

### BRIEF DESCRIPTION OF THE DRAWINGS

FIG. 1 documents physical-chemical parameters from an 80 liter fermentation for production of rSEB. Glutamate, OD 600, pH, DO and CO₂ were monitored throughout the production. The temperature range was 36-38° C. The agitation rate was increased from 185 to 500 rpm by the 3.5 hour time point.

FIGS. 2A and 2B depict Western blot data demonstrating relative rSEB expression and solubility from point of induction to four hours post induction. rSEB was observed at low levels in the control sample but steadily accumulated in the soluble fraction of the cell from one hour post induction until harvest. FIG. 2A depicts the results for cell lysate supernatant; Lane 1: SEE BLUE MOLECULAR WEIGHT MARKER; Lane 2: purified SEB standard; Lane 3 before induction (10 μl); Lane 4: After induction 0 hr (10 μl); Lane 5: After induction 1 hr (10 μl); Lane 6: After induction 2 hr (10 μl); Lane 7: After induction 3 hr (10 μl); Lane 8: After induction 4 hr (10 μl). FIG. 2B depicts the results for cell lysate pellet; Lane 1: SEE BLUE MOLECULAR WEIGHT MARKER; Lane 2: purified SEB standard; Lane 3 before induction (10 μl); Lane 4: After induction 0 hr (10 μl); Lane 5: After induction 1 hr (10 μl); Lane 6: After induction 2 hr (10 μl); Lane 7: After induction 3 hr (10 μl); Lane 8: After induction 4 hr (10 μl).

FIG. 3 depicts a flow chart of the upstream recovery process. Tangential flow filtration was used to remove cell and cell debris. Preliminary evaluation showed that the rSEB was completely retained by the 30 kDa filter. Contemporaneous studies have shown that the rSEB remains unchanged after over 12 months when stored at −70§C as an ammonium sulfate precipitate.

FIGS. 4A and 4B depict an SDS-PAGE analysis of rSEB after upstream purification steps. FIG. 4A, Lane 1=molecular weight standards; Lanes 2-5: 20 μg, 10 μg, 5 μg and 2.5 μg, respectively, of the supernatant fraction following 45% ammonium sulfate precipitation; Lane 6: blank; Lanes 7-10: 20 μg, 10 μg, 5 μg and 2.5 μg, respectively, of the rSEB-enriched 75% ammonium sulfate precipitate. FIG. 4B depicts results for rSEB following HIC purification. Lane 1: resuspended 75% ammonium sulfate material (load solution); Lane 2: rSEB-enriched flow-through fraction; Lane 3: low-salt strip of adsorbed, rSEB-depleted fraction. 20 μg of each fraction were analyzed. Samples were diluted in a low-salt buffer and the protein content was determined by the Pierce COOMASSIE® PLUS assay using Bovine Serum Albumin as a reference standard.

FIG. 5 is a chromatogram of rSEB purified on POROS® 20 HS ion-exchange resin. Thawing of the frozen, intermediate-bulk product resulted in the formation of trace amounts of rSEB dimer. These were easily and reproducibly isolated from the main monomer pool with minimal loss of intact product.

FIGS. 6A through 6D demonstrate the purity of final purified bulk rSEB following polishing on SUPERDEX-75. FIG. 6A: MALDI-TOF. FIG. 6B: SDS-PAGE. Lane 1: molecular weight standards; Lanes 2-4: 25 μg, 10 μg, 20 μg, respectively, under reducing conditions; Lanes 8-10: 25 μg, 10 μg, 20 μg, respectively, under non-reducing conditions. FIG. 6C: Reversed-phase HPLC. FIG. 6D: Capillary Zonal Electrophoresis. Samples were prepared and analyzed as described below. No significant impurities were identified by any of these methods.

FIG. 7 demonstrates purity of final, purified rSEB bulk product by SEC-HPLC. 25 μg of purified rSEB was analyzed on a Tosohaas G3000SW HPLC column. Detection was at OD280 nm. Peak purity was greater than 99%.

FIG. 8 depicts capillary isoelectric focusing (cIEF) purity analysis of final purified bulk rSEB. Samples were prepared and analyzed as described here in for analytical methods. Peaks at 9.73 and 15.41 minutes correspond to pH 8.4 and 10.4 internal standards. The pI of rSEB was calculated to be 9.0-9.1. There was no evidence of rSEB isoforms and no significant change was observed in this pattern after 12 months of storage at −70° C.

### DESCRIPTION OF THE SPECIFIC EMBODIMENTS

For the production of many polypeptides and proteins, recombinant DNA techniques have become the method of choice, because large quantities of exogenous proteins can be expressed in bacteria and other host cells. Producing recombinant proteins involves transfecting host cells with DNA encoding the protein and growing the cells under conditions favoring expression of the recombinant protein. The prokaryote E. coli is a favored host in this context because it can be made to produce recombinant proteins in high yields at low cost. Numerous U.S. patents on general bacterial expression of DNA encoding proteins exist, including U.S. Pat. No. 4,565,785 on a recombinant DNA molecule comprising a bacterial gene for an extracellular or periplasmic carrier protein and non-bacterial gene; U.S. Pat. No. 4,673,641 on co-production of a foreign polypeptide with an aggregate-forming polypeptide; U.S. Pat. No. 4,738,921 on an expression vector with a trp promoter/operator and trp LE fusion with a polypeptide; U.S. Pat. No. 4,795,706 on expression control sequences to include with a foreign protein; and U.S. Pat. No. 4,710,473 on specific circular DNA plasmids (each of the foregoing references is incorporated herein by reference).

Genetically engineered biopharmaceuticals, including recombinant bacterial SAgs, are typically purified from a supernatant containing a variety of diverse host cell contaminants. As noted above, producing a recombinant protein in this manner involves transfecting host cells with DNA encoding the protein and growing the cells under conditions favoring expression of the recombinant protein. Bacterial SAgs have been reportedly purified from such recombinant expression systems to varying extents with varying degrees of effort and success using a number of different methods (see, for example, Ulrich et al., Vaccine 16:1857-1864, 1998, incorporated herein by reference). However, preparative isolation of a recombinant SAg, and in particular a recombinant SEB protein, resulting in pharmaceutical purity and high yield, has heretofore eluded the art.

The increasing need for highly efficient recombinant production systems to manufacture large quantities of pure, recombinant bacterial SAg product (e.g., clinical grade product, essentially free of undesirable or harmful contaminants such as DNA and endotoxins) follows the development of new vaccine technologies to prevent or treat diseases and other conditions (including those potentially caused by SAg biowarfare agents) mediated by SAgs. Among these emergent technologies are those that rely on the use of vaccines comprising a bacterial SAg modified by limited site-directed mutagenesis within conservative receptor-binding motifs. As noted above, these structurally conserved motifs are common among all bacterial SAgs and are useful for construction of highly effective recombinant SAg vaccine agents (Ulrich et al., Vaccine 16:1857-1864, 1998; Bavari et al., J. Infect. Dis. 174:338-345, 1996; and Ulrich et al., Nature Struct. Biol. 2:554-560, 1995).

One important bacterial SAg within the instant invention is a native staphylococcal enterotoxin B (SEB) antigen that has been cloned and modified from the native SEB toxin at three sites (Ulrich et al., Vaccine 16:1857-1864, 1998, incorporated herein by reference). The three mutations in this proposed vaccine product have been shown to eliminate measurable binding of the recombinant SEB (rSEB) to HLA-DR-bearing cells in vitro. In addition, the three amino acid substitutions in this modified toxin were selected to essentially eliminate the possibility of a random mutation occurring within the recombinant construct that might result in the reversion of the protein to toxic form.

In this exemplary SEB mutant, amino acid substitutions at position 89 (from tyrosine to alanine), position 45 (from leucine to arginine), and position 94 (from tyrosine to alanine) were selected after testing several single site-directed mutants in the MHC-binding site for loss of activity. Structural modeling had suggested that these substitutions would have minimal effect on protein conformation, thereby retaining antigenicity (Ulrich et al., Vaccine 16:1857-1864, 1998). The triple mutant has shown no SAg-like properties, such as induction of cytokine synthesis or anergy induction in human or non-human primate (NHP) lymphocytes, and no intoxication in mice or non-human primates. The recombinant molecule has 244 amino acids, a calculated pI of 8.95, an extinction coefficient of 30,130 g/mol and a molecular mass of 28.4 kDa. The modified SEB gene (designated B899445C) encoding the mutated, nontoxic form of the SEB protein has been engineered in a pET24b (+) prokaryotic expression vector system. The expression vector uses the T7 phage promoter and is expressed in an E. coli BL21 (DE3) strain selected for with the antibiotic, kanamycin.

The instant disclosure presents unexpected results by providing novel methods and compositions for efficiently manufacturing clinical grade, recombinant bacterial SAgs, including rSEBs such as the rSEB encoded by the recombinant B899445C gene, on a large scale. The methods and compositions of the invention are applicable to starting material from varying sources, including fermentation broth, lysed bacterial or mammalian cells.

The following abbreviations are used herein below to facilitate understanding of the invention.

AP alkaline phosphatase

BCA bicinchoninic acid

CDC Centers for Disease Control

CFU colony-forming unit

CGMP current good manufacturing practices

cIEF capillary isoelectric focusing

CV column volumes

CZE capillary zonal electrophoresis

DO dissolved oxygen

EDTA ethylenediaminetetraacetic acid

EU endotoxin unit

FDA Food and Drug Administration

FT flow throughfraction

HIC hydrophobic interaction chromatography

HLA human leukocyte antigen

HPLC high-performance liquid chromatography

HP-SEC high-pressure size-exclusion chromatography

i.d. inner diameter

IPTG isopropyl-β-D-thiogalactopyranoside.

LD₅₀ lethal dose to kill 50% of test animals

LOD limit of detection

LPS lipopolysaccharide

MALDI-TOF matrix-assisted laser desorption/ionization-time-of-flight

MCB master cell bank

MHC major histocompatibility complex

NBT-BCIP nitroblue tetrazoleum-5-bromo-4-chloro-3-indolyl phosphate

NHP nonhuman primate

PBS phosphate-buffered saline

pI isoelectric point

rSEB recombinant Staphylococcul enterotoxin B

SAg superantigen

SDS-PAGE sodium dodecyl sulfate-polyacrylamide gel electrophoresis

SE staphylococcal enterotoxin

SLMP standard liter per minute

SYG media soytone-yeast extract-glycerol

TCEP tris(2-carboxyethyl) phosphine hydrochloride

TCR T-cell antigen receptor

TFA trifluoroacetic acid

TFF tangential flow filtration

TSA tryptic soy agar

TSAG tryptic soy agar plus glucose

UV ultraviolet

Bacterial SAgs for use within the instant invention can be prepared by any means, but they are typically prepared recombinantly. A nucleic acid molecule coding for the recombinant SAg can be obtained from several sources, for example, through chemical synthesis using the known DNA sequence or by the use of standard cloning techniques known to those skilled in the art. cDNA clones carrying the SAg coding sequence can be identified by use of oligonucleotide hybridization probes specifically designed based on the known sequence of a particular SAg of interest, or based on a known consensus sequence elements shared among different SAgs.

Upon obtaining a molecule having an SAg coding sequence, the molecule is inserted into a cloning vector appropriate for expression in a selected host cell. The cloning vector is constructed so as to provide the appropriate regulatory functions required for the efficient transcription, translation and processing of the coding sequence.

If the bacterial SAg is prepared recombinantly, suitable host cells for expressing the DNA encoding the SAg are prokaryote, yeast, or higher eukaryotic cells. Suitable prokaryotes for this purpose include bacteria such as archaebacteria and eubacteria. Preferred bacteria are eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella; Bacilli such as B. subtilis and B. licheniformis (e.g., B. licheniformis 41P disclosed in DD 266,710 published Apr. 12, 1989, incorporated herein by reference); Pseudomonas such as P. aeruginosa; Streptomyces; Azotobacter; Rhizobia; Vitreoscilla; and Paracoccus. Suitable E. coli hosts include E. coli W3110 (ATCC 27,325), E. coli 94 (ATCC 31,446), E. coli B, and E. coli X1776 (ATCC 31,537). These examples are considered illustrative rather than limiting.

Mutant cells of any of the above-mentioned bacteria may also be employed. It is, of course, necessary to select the appropriate bacteria taking into consideration replicability of the replicon in the cells of a bacterium. For example, E. coli, Serratia, or Salmonella species can be suitably used as the host when well known plasmids such as pBR322, pBR325, pACYA177, or pKN410 are used to supply the replicon. E. coli strain W3110 is a commonly used host or parent host because it is a routinely used host strain for recombinant DNA product fermentation. Typically, the desired host cell secretes minimal amounts of proteolytic enzymes. For example, strain W3110 may be modified to effect a genetic mutation in the genes encoding proteins endogenous to the host, with examples of such hosts including E. coli W3110 strain 1A2; E. coli W3110 strain 9E4; E. coli W3110 strain 27C7 (ATCC 55,244); E. coli W3110 strain 37D6; E. coli W3110 strain 40 B4; and an E. coli strain having mutant periplasmic protease such as disclosed in U.S. Pat. No. 4,946,783 issued Aug. 7, 1990 (incorporated herein by reference).

As noted above, a mutant form of SEB has been expressed in E. coli. The isolation and sequence of the gene encoding the rSEB (designated B899445C) and its expression as a heterologous protein in E. coli was previously described (Ulrich et al., Vaccine 16: 1857-1864, 1998, incorporated herein by reference). In addition to prokaryotes, eukaryotic microbes such as filamentous fungi or yeast are suitable expression hosts for bacterial SAgs. Saccharomyces cerevisiae, or common baker's yeast, is the most commonly used among lower eukaryotic host microorganisms. However, a number of other genera, species, and strains are commonly available and useful herein, such as Schizosaccharomyces pombe (Beach and Nurse, Nature 290:140, 1981; EP 139,383 published May 2, 1985; Kluyveromyces hosts (U.S. Pat. No. 4,943,529; Fleer et al., Bio/Technology 9:968-975, 1991) such as, e.g., K. lactis (Louvencourt et al., J. Bacteriol. 154:737-742 1983), K. fragilis (ATCC 12,424), K bulgaricus (ATCC 16,045), K. wickeramii (ATCC 24,178), K. waltii (ATCC 56,500), K. drosophilarum (ATCC 36,906; Van den Berg et al., Bio/Technology 8:135, 1990), K. thermotolerans, and K. marxianus; yarrowia (EP 402,226); Pichia (EP 183,070; Sreelrishna et al., J. Basic Microbiol. 28:265-278, 1988); Candida; Trichoderma reesia (EP 244,234); Neurospora crassa (Case et al., Proc. Natl. Acad. Sci. USA 76:5259-5263, 1979); Schwanniomyces such as Schwanniomyces occidentalis (EP 394,538 published Oct. 31, 1990); and filamentous fungi such as, e.g., Neurospora, Penicillium, Tolypocladium (WO 91/00357 published Jan. 10, 1991), and Aspergillus hosts such as A. nidulans (Ballance et al., Biochem. Biophys. Res. Commun., 112: 284-289, 1983; Tilbum et al., Gene 26:205-221, 1983; Yelton et al., Proc. Natl. Acad. Sci. USA 81:1470-1474, 1984) and A. niger (Kelly et al., EMBO J. 4:475-479, 1985) (each of the foregoing publications are incorporated herein by reference).

Suitable host cells appropriate for the expression of the DNA encoding the SAg can also be derived from multicellular organisms. Examples of useful invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. See, e.g., Luckow et al., Bio/Technology 6:47-55, 1988; Miller et al., in Genetic Engineering, Setlow, J. K. et al., eds., Vol. 8, Plenum Publishing, 1986, pp. 277-279; and Maeda et al., Nature 315:592-594, 1985), each incorporated herein by reference. A variety of viral strains for transfection are publicly available, e.g., the L-1 variant of Autographa californica NPV and the Bm-5 strain of Bombyx mori NPV, and such viruses may be used herein, particularly for transfection of Spodoptera frugiperda cells.

Plant cell cultures of cotton, corn, potato, soybean, petunia, tomato, and tobacco can also be utilized as hosts. Typically, plant cells are transfected by incubation with certain strains of the bacterium Agrobacterium tumefaciens, which have been previously manipulated to contain the DNA encoding the SAg. During incubation of the plant cell culture with A. tumefaciens, the DNA encoding the SAg is transferred to the plant cell host such that it is transfected, and will, under appropriate conditions, express the DNA encoding the SAg. In addition, regulatory and signal sequences compatible with plant cells are available, such as the nopaline synthase promoter and polyadenylation signal sequences (Depicker et al., J. Mol. Appl. Gen. 1:561, 1982, incorporated herein by reference). In addition, DNA segments isolated from the upstream region of the T-DNA 780 gene are capable of activating or increasing transcription levels of plant-expressible genes in recombinant DNA-containing plant tissue (EP 321,196 published Jun. 21, 1989, incorporated herein by reference).

Examples of useful mammalian host cell lines are monkey kidney CV1 line transformed by SV40 (COS-7, ATCC CRL 1651); human embryonic kidney line (e.g., 293 or 293 cells subcloned for growth in suspension culture (Graham et al., J. Gen Virol. 36:59, 1977); baby hamster kidney cells (13HK, ATCC CCL 10); Chinese hamster ovary cells/DHFR (CHO, Urlaub and Chasin, Proc. Natl. Acad. Sci. USA 77:4216, 1980); mouse Sertoli cells (e.g., TM4, Mather, Biol. Reprod. 23:243-251, 1980); monkey kidney cells (e.g. CV1, ATCC CCL 70); African green monkey kidney cells (e.g. VERO-76, ATCC CRL-1587); human cervical carcinoma cells (e.g., HeLa, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., HepG2, ATCC HB 8065); mouse mammary tumor (MMT 060562, ATCC CCL51); TRI cells (Mather et al., Ann. N.Y. Acad. Sci. 383:44-68, 1982); MRC 5 cells; FS4 cells; and the like.

Host cells are transfected and preferably transformed with the above-described expression or cloning vectors and cultured in conventional nutrient media modified as appropriate for inducing promoters, selecting transformants, or amplifying the genes encoding the desired sequences. Transfection refers to the taking up of an expression vector by a host cell whether or not any coding sequences are in fact expressed. Numerous methods of transfection are known to the ordinarily skilled artisan, for example, CaPO₄ and electroporation. Successful transfection is generally recognized when any indication of the operation of the vector occurs within the host cell.

Transformation means introducing DNA into an organism so that the DNA is replicable, either as an extrachromosomal element or by chromosomal integration. Depending on the host cell used, transformation is done using standard techniques appropriate to such cells. The calcium treatment employing calcium chloride, as described in Protocol 25, at page 1.116 of Sambrook and Russell, (Molecular Cloning: A Laboratory Manual Third Edition, Cold Spring Harbor Laboratory Press, Cold Spring Harbor, N.Y., 2001, incorporated herein by reference), or electroporation (Protocol 26, Sambrook and Russell, supra) is generally used for prokaryotes or other cells that contain substantial cell-wall barriers. Infection with Agrobacterium tumefaciens can be used for transformation of certain plant cells, as described by Shaw et al., Gene 23:315, 1983, and WO 89/05859 published Jun. 29, 1989 (each incorporated herein by reference). In addition, plants may be transformed using ultrasound treatment as described in WO 91/00358 published Jan. 10, 1991 (incorporated herein by reference).

For mammalian cells without such cell walls, the calcium phosphate precipitation method of Graham et al. (Virology 52:456-457, 1978, incorporated herein by reference) is preferred. General aspects of mammalian cell host system transformations have been described by, for example, Axel in U.S. Pat. No. 4,399,216 issued Aug. 16, 1983 (incorporated herein by reference). Transformations into yeast are typically carried out according to the method of Van Solingen et al., J. Bact. 130:946-947, 1977 and Hsiao et al., Proc. Natl. Acad. Sci. USA 76:3829-3833, 1979. However, other methods for introducing DNA into cells, such as by nuclear microinjection, electroporation, bacterial protoplast fusion with intact cells, or polycations, e.g., polybrene, polyomithine, and the like, can also be used. For various techniques for transforming mammalian cells, see for example, Keown et al., Methods Enzymol. 185:527-537, 1990, and Mansour et al., Nature 336:348-352, 1988 (each incorporated herein by reference).

If prokaryotic cells are used to produce the bacterial SAg, they are cultured in suitable media in which the promoter can be constitutively or artificially induced as described generally, e.g., in Sambrook and Russell, Molecular Cloning: A Laboratory Manual, Third Edition (Cold Spring Harbor Laboratory Press, Cold Spring Harbor, N.Y., 2001, incorporated herein by reference). Any necessary supplements besides carbon, nitrogen, and inorganic phosphate sources can also be included at appropriate concentrations introduced alone or as a mixture with another supplement or medium such as a complex nitrogen source.

If mammalian host cells are used to produce the rSAg, they may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Methods Enzymol. 58:44-93, 1979; Barnes and Sato, Anal. Biochem. 102:255-270, 1980; U.S. Pat. Nos. 4,767,704; 4,657,866; 4,927,762; 5,122,469; or 4,560,655; WO 90/03430; WO 87/00195; or U.S. Pat. No. Re. 30,985 (each incorporated herein by reference) can be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as Gentamycin, kanamycin, streptomycin and/or penicillin), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those generally used with the host cell selected for expression, and will be apparent to the ordinarily skilled artisan.

In general, principles, protocols, and practical techniques for maximizing the productivity of in vitro mammalian cell cultures can be found in Mammalian Cell Biotechnology: A Practical Approach, M. Butler, ed. (IRL Press at Oxford University Press, Oxford, 1991, incorporated herein by reference). The above process can be employed whether the SAg is produced intracellularly, produced in the periplasmic space, or directly secreted into the medium.

In certain detailed embodiments of the invention, the recombinant SAg gene is incorporated in E. coli host cells and used to generate a Master Cell Bank, from which seed stocks for the production of the recombinant SAg protein are derived. In one exemplary embodiment, an original seed stock of E. coli containing the rSEB gene (899445C) on plasmid pET 24b(+) is used to prepare a Master Cell Bank (MCB). The MCB is typically generated in 1 liter, triple-baffled shake flasks and batched with 24 g/L of yeast extract, 12 g/L of soytone, 4 ml/L of glycerol, 2.31 g/L of KH₂PO₄, 12.5 g/L of K₂HPO₄, and 50 μg/mL of kanamycin, and are inoculated with single-colony isolates (e.g., grown on Tryptic Soy Agar (TSA) plates). The flasks are placed on a rotary shaker at about 200 rpm and incubated overnight at 37° C. to an OD₆₀₀ of approximately 14. The flasks are then pooled, and glycerol is added to a final concentration of 13%. The cells are aliquoted at 1 ml in a 2-ml cyrovial and subjected to a slow-rate freeze. Vials are tested for purity, gram stain, biological type, and rSEB gene-insert sequence integrity. This process yields an average cell count of about 3.98×10⁸ CFU/ml in the MCB.

The Master Cell Bank is expanded typically through two seed batches prior to expansion to form the production culture that will be induced for SAg expression. During the seed phase the cultures are expanded and monitored for health and viability prior to induction. Typically, the fermentation procedure for recombinant E. coli is conducted in two stages. In a first stage, shake flasks are batched with sterile seed medium (e.g., 24 g/L yeast extract, 12 g/L soytone, 4 ml/L glycerol, 2.31 g/L KH₂PO₄, 12.5 μL K₂HPO₄, and 50 μg/ml kanamycin), and are inoculated aseptically with pooled material (e.g., at 1% v/v) from the MCB. First-stage seed flasks are placed on a rotary shaker at about 250 rpm and incubated at 37° C. One of the flasks is selected to obtain time-course samples at one-hour intervals. The time-course samples are processed for OD₆₀₀, pH, growth on Tryptic Soy Agar plus glucose (TSAG) plates, and observed by wet mount. Based on the growth curve (OD₆₀₀ values vs time), the first-stage seed culture is typically ready to scale to a second-stage seed culture when late log phase growth is observed. This stage is indicated by a measure OD₆₀₀ of about 5 to 15, more typically an OD₆₀₀ of 8±2 is measured.

In the second stage, typically, three 2000-ml, tripled baffled, shake flasks are batched with 480 ml of sterile seed medium. Each second-stage seed flask is inoculated aseptically with about 9.6 ml (2% v/v) of the first-stage seed culture. All second-stage seed flasks are placed on a rotary shaker at about 250 rpm and incubated at 37° C. One of the flasks is selected to obtain time-course samples at one-hour intervals. The time-course samples are processed for OD₆₀₀, pH, growth on Trytic Soy Agar plus glucose (TSAG) plates, and observed by wet mount. Criteria to scale the second-stage seed to the production fermenter are based on the resulting growth curve (late log phase or early stationary phase) and a requirement to reach a specified OD₆₀₀ typically 5 to 15, more typically 8.0±2.0.

The production stage is typically, but not limited to being, conducted in an 80-L fermenter with a working volume of 48 Liters. The production fermenter is batched with 24 g/L of yeast extract, 12 g/L of soytone, 4 g/L of glycerol, 2.31 g/L of KH₂PO₄, 12.5 g/L of K₂HPO₄, 0.1% v/v of P2000 antifoam, and 50 μg/ml kanamycin. The fermenter is maintained at a temperature of 37° C., an agitation rate of about 177 to about 461 rpm, an aeration rate of about 16 to about 58 standard liter per minute (slpm), and a vessel pressure of 3 to 7 psig. The percent dissolved oxygen (% DO) within the fermenter is maintained at >20% by first adjusting the sparging and then the agitation rate incrementally. Time-course samples are obtained aseptically from the fermentation vessel at least at about one-hour intervals and processed for OD₆₀₀, pH, glutamate concentration, growth on TSAG plates, and observation on wet mount. The culture is aseptically fed about 2.4 L of a sterile 10× nutrient feed (24 g/L yeast extract, 12 g/L soytone, 4 g/L glycerol, 9.6 L purified water) at a rate of about 50 ml/minute when the OD₆₀₀ reaches about 8±2, or the amount of glutamate in the production culture is less than about 50% of the original glutamate concentration at inoculation. The infusion of 10× nutrient feed is continued as long as the DO levels are greater than 20%. The culture is typically induced with filter-sterilized, dioxane-free IPTG at a 0.1 to 3.0 mM final concentration when the OD₆₀₀ reached about 5 to about 20. More typically the final concentration ranges from 0.5 mM to about 20 mM and even more typically a range of 1±0.2 mM dioxane-free IPTG is used. Induction of the culture can also be typically carried out when the OD₆₀₀ reaches about 10 to about 15 (12.5±2.5).

Immediately after IPTG induction, the culture is typically fed with about 2.4 L of a filter-sterilized 20× yeast nitrogen base solution (2 g/L yeast nitrogen base, 2.4 L purified water). The culture is next prepared for harvest, at about 4 h after IPTG induction. Prior to harvest, a final sample may be obtained aseptically and processed for TSAG plate, OD₆₀₀, pH, glutamate concentration, and DNA sequencing. The sample is also observed by wet mount. In preparation for harvest, the fermenter is typically chilled to about 10° C., the agitation rate reduced to about 55 rpm, and the aeration rate adjusted to about 16 slpm.

The novel fermentation methods and compositions of the present invention provide a surprisingly high yield of soluble SAg. In particular, the use of a yeast extract-based culture medium, and separately the addition of trace elements (e.g. one or more trace elements selected from NH₄SO₄, ZnSO₄.7H₂O, CuSO₄.5H₂O, MnSO₄.H₂O, FeCl₃.6H₂O, CoCl₂.6H₂O, Na₂MoO₄.2H₂O) were found to unexpectedly improve the final yield. Increased yields based on these parameters may be up to two-fold, five-fold, ten-fold or greater compared to control fermentation batches fed with a different medium and/or lacking selected trace element(s). Also noted herein is the finding that glucose suppressed the final OD₆₀₀ in exemplary E. coli cultures and should therefore be omitted from the final media formulation.

In further detailed aspects of the invention, the timing of induction and feeding may be modified to provide enhanced levels of soluble protein from the fermentation. For example, when transformed E. coli cells are cultured at 37° C. and induced with 1 mM IPTG, approximately 70% of the protein is obtained in the soluble fraction, while only 30% remains in the insoluble fraction as inclusion bodies. This compares very favorably to other expression fermentation systems (where the soluble protein fraction is typically below 30%, often as low as 10-15% or lower). These results mark additional unexpected results provided by the fermentation methods of the present invention, which generally yield a soluble SAg fraction of 30-50%, typically more than 50%, often up to 70% or greater (e.g., 90% or greater). For example, in one E. coli fermentation example discussed below, the ability to drive more SAg product into the soluble fraction is attributed to modifying induction conditions. Thus, in a 20 liter pilot fermentation greater than 90% of an rSEB product was expressed as a soluble protein when the culture was induced at an OD₆₀₀ of 10-15 with 1 mM IPTG at 37° C. for 4 h. Typical induction OD₆₀₀ values to increase soluble protein yield in this context range between about 5.0 and about 20.0, but more typically range between about 12.5±2.5.

Additional advantages are provided within the fermentation methods of the present invention which involve the use of alternative nitrogen sources than those routinely used in the art derived from bovine or other animal products. In one example, peptones from non-animal sources were demonstrated to support growth. In more detailed aspects, tryptase was found to be superior to other nitrogen sources evaluated, while soytone was also shown to be satisfactory for supporting large-scale fermentation.

In related embodiments of the invention, post-fermentation recovery is performed using equipment cooled with chilled water (<55° F.). The fermented broth is transferred to a chilled tank and concentrated. The broth is typically concentrated from a starting volume to a calculated cell density of approximately 250 to 300 g/L. Residual soluble media components may be removed, for example by diafiltration. EDTA concentration in the resulting preparative mixture may be adjusted, typically up to about 20 mM.

Further recovery procedures from rSAg production systems typically involves disruption of the host cells. This can be achieved by any appropriate technique, for example, mechanical methods, e.g., a Manton-Gaulin press, a French press, or a sonic oscillator, or by chemical or enzymatic methods. Examples of chemical or enzymatic methods of cell disruption include spheroplasting, which entails the use of lysozyme to lyse the bacterial wall (Neu et al., Biochem. Biophys. Res. Comm. 17:215, 1964, incorporated herein by reference), and osmotic shock, which involves treatment of viable cells with a solution of high tonicity and with a cold-water wash of low tonicity to release the polypeptides (Neu et al., J. Biol. Chem. 240:3685-3692, 1965, incorporated herein by reference). A third method, described in U.S. Pat. No. 4,680,262 (incorporated herein by reference), involves contacting the transformed bacterial cells with an effective amount of a lower alkanol having 2 to 4 carbon atoms for a time and at a temperature sufficient to kill and lyse the cells.

After the cells are disrupted, the resulting suspension may be centrifuged to remove cellular debris, inclusion bodies and other contaminants. In certain embodiments, this step is carried out at about 500 to 15,000×g, more typically at about 12,000×g, in a standard centrifuge for a sufficient time that depends on volume and centrifuge design, usually about 10 minutes to 0.5 hours. The resulting pellet contains substantially all of the insoluble polypeptide fraction, but if the cell disruption process is not complete, it may also contain intact cells or broken cell fragments. Completeness of cell disruption can be assayed by resuspending the pellet in a small amount of the same buffer solution and examining the suspension with a phase-contrast microscope.

In alternative embodiments, rSAg proteins are isolated from the periplasmic space by solubilization in a suitable buffer. This procedure can be in-situ solubilization involving direct addition of reagents to the fermentation vessel after the protein has been produced recombinantly, thereby avoiding extra steps of harvesting, homogenization, and centrifugation to obtain the protein. The remaining particulates can be removed by centrifugation or filtration, or combinations thereof. Alternatively, one may use a multiple-phase isolation/extraction system for purifying SAg protein from the remaining particulates, as described in U.S. Pat. No. 5,407,810.

In various embodiments of the invention, the crude preparation of SAg obtained from the host cells (e.g., by cell disruption) is initially subjected to hydrophobic interaction chromatography (HIC) as an initial purification step to separate the recombinant SAg from contaminants such as other proteins, proteolytic degradation products or multimeric forms of SAg, endotoxins, and the like. Optionally, the crude preparation of SAg obtained from the host cells may be subjected to preliminary processing or purification steps prior to HIC. For example, the SAg protein may be solubilized from aggregate form (inclusion bodies) using known methods, which may in turn require refolding of the protein into an active conformation (see, e.g., U.S. Pat. No. 5,663,304, incorporated herein by reference) to maximize the yield of correctly folded protein and the ratio of correctly folded, monomeric protein (e.g., as determined by radioimmunoassay (RIA) or high pressure liquid chromatography (HPLC), and/or mass balance).

Other optional, preliminary purification steps prior to HIC may include any of a variety of separation methods, for example, fractionation on immunoaffinity or ion-exchange columns; ethanol precipitation; reversed-phase HPLC; chromatography on silica; chromatography on an ion-exchange resin such as S-SEPHAROSE and DEAE; chromatofocusing; SDS-PAGE; ammonium sulfate precipitation; and/or gel filtration using, for example, SEPHADEX G-75.

In certain detailed embodiments, bacterial cell lysates are preliminarily clarified by diafiltration. A subsequent lysate filtrate is then optionally subjected to tangential flow filtration (TFF). Typically, the TFF retentate, or an unprocessed, crude SAg preparation, is subjected to differential ammonium sulfate precipitation, e.g., by adding solid ammonium sulfate to a desired saturation. This desired level of saturation is important in that it controls the retention, or loss, of SAg from the preparation. In certain embodiments of the invention, the level of ammonium sulfate saturation is between about 30-60%, often between about 40-50%, and preferably about 45% or less. The pH may be monitored during this procedure and maintained between about 6.5 and 8.0, preferably between about 7.0 and 7.4 (e.g., by adding 0.5 M ammonium hydroxide). The suspension can then be stored (e.g., for 4 h at 2 to about 8° C., or more), after which the precipitate is recovered (e.g., by means of bottle centrifugation in a 2 to 8° C. refrigerated centrifuge). In more detailed embodiments, the supernatant is decanted into a sterile, depyrogenated container, and the pellets were discarded. Solid ammonium sulfate is then added to the 45% precipitation supernatant to a final concentration of at least 55%, preferably 60% or greater, and most preferably about 75% saturation. The pH is monitored during this procedure and maintained between about 6.5 and 8.0, preferably between about 7.0 and 7.4 (e.g., by adding 0.5 M ammonium hydroxide). This suspension may also be stored (e.g., for a minimum of 4 h at 2 to 8° C.), after which the precipitate is recovered. The supernatant at this stage is decanted as waste, and the recovered pellets can be stored at or below −60° C. In certain embodiments, the rSAg stored under these conditions (e.g., at −70° C.) is stable (e.g., does not exhibit degradation products or, alternatively, possesses at least 85% of the biological activity as determined by various known assays as a comparable amount of rSAg or native SAg not subjected to the storage) for up to twelve-eighteen months or more, often for two to several years or more.

Prior to HIC, the frozen ammonium sulfate pellets are solubilized, for example by the addition of 25 mM sodium phosphate buffer (e.g., at pH 7.0). Pellets are completely solubilized by gentle stirring. Samples may be removed for determination of protein content, e.g., by bicinchoninic acid (BCA) assay, and based on the protein concentration the solubilized pellets are diluted (e.g., by the addition of 1.0 M ammonium sulfate, 25 mM sodium phosphate buffer, pH 7.0), to obtain a final protein concentration of about 1.5 mg/mL. Solubilized pellets were then filtered into a sterile, chilled, pyrogen-free sterile vessel.

In certain preferred aspects of the invention, a starting load sample comprising a recombinant SAg is subjected to HIC. The starting load sample can, for example, be any end product of a recombinant SAg expression process, for example an end product of fermentation of E. coli transformed to express a rSAg gene. As such, the starting load material can be a crude or partially purified cell lysate, or a sample or extract of a culture medium if the rSAg product is secreted in the expression system. In certain exemplary embodiments, the starting load material is obtained by solubilizing an ammonium sulfate pellet comprising a recombinant SAg in a precipitated, storage stable form, as described above.

HIC involves sequential adsorption and desorption of protein from solid matrices mediated through non-covalent hydrophobic bonding. Generally, a starting load mixture of subject (e.g., rSAg) and contaminant (e.g., endotoxin, and the like) molecules in a high salt buffer is loaded on the HIC column. The salt in the buffer interacts with water molecules to reduce the solvation of the molecules in solution, thereby exposing hydrophobic regions in the subject and contaminant molecules which may consequently be adsorbed by the HIC column. The more hydrophobic the molecule, the less salt needed to promote binding. Usually, a decreasing salt gradient is used to elute samples from the column. As the ionic strength decreases, the exposure of the hydrophilic regions of the molecules increases and molecules elute from the column in order of increasing hydrophobicity. Sample elution is also achieved by the addition of mild organic modifiers or detergents to the elution buffer. HIC is reviewed in, for example, Protein Purification, 2d Ed., Springer-Verlag, New York, pgs 176-179 (1988), incorporated herein by reference.

The strength of the association between a protein and a matrix depends on several factors, including the size and hydrophobic character of the immobilized functional group, the polarity and surface tension of the surrounding solvent, and the hydrophobicity of the protein. The binding capacity of HIC matrices tends to be low due to the need for the immobilized hydrophobic ligand to be widely spaced. Further, the capacity of a medium for a given protein varies inversely with the level of hydrophobic impurities in the sample preparation. In order to resolve a desired protein from variants and other impurities while simultaneously maximizing capacity, it is necessary to identify a suitable HIC solid-phase medium as well as suitable mobile phases for load, wash, and elution.

Using HIC, a variety of mobile phase conditions can be used to wash and differentially elute subject and contaminant molecules. These mobile phases can contain 0.5 several different chemical species that influence the association between a subject or contaminant molecule and the stationary phase in different ways. According to the present invention, subject or contaminant molecules can be resolved on a “IC column by decreasing salt gradients or step-wise decrease, for example of mobile-phase salt, e.g., ammonium sulfate, NaCl concentration, or acetate concentration. Salts can influence the binding of a subject or contaminant molecule to the resin by modulating the surface tension of the mobile phase. Other agents that can be used to modulate surface tension are sodium citrate and tetramethyl ammonium chloride. Subject and contaminant molecules can also be resolved during column HIC by eluting bound molecules with increasing gradients or step-wise increase in concentration of relatively polar organic solvents. Examples of suitable solvents in this regard include ethanol, acetonitrile, and propanol. The strength of the association subject and contaminant molecules and the HIC matrix also depends on the mobile-phase pH, with neutral conditions preferred.

Separation of subject and contaminant molecules can also be obtained by simultaneously varying several properties of the mobile phase during gradient or step-wise elution. For example, a mobile phase that simultaneously varies in salt concentration and a polar solvent concentration during elution may provide better resolution than when only the salt concentration is varied. In certain embodiments, a lowering of the salt concentration is used to elute and separate subject and contaminant molecules. In order to achieve elution, the salt concentration in the elution buffer is typically lower than that in the loading buffer, but it can be the same concentration when compensated for with organic solvent. In addition, the use of organic solvent can confer another advantage, because the addition of an organic solvent may improve the elution pattern by resulting in narrower peak profiles. In addition to ethanol, other organic solvents can be used, including propanol, isopropanol, and lower alkylene glycols, such as propylene glycol, ethylene glycol and hexylene glycol, and the like. Typically, the organic solvent is incorporated at about 5 to about 25% (v/v), more typically about 5 to about 20% (v/v). The elution with organic solvent can be either gradient or step-wise. The pH range is typically near neutral to slightly acidic, for example, from pH 5 to 8, more typically pH 6 to 8, pH 6.5 to 7.5, and most commonly pH 7. Any of the buffers discussed herein, including 3-(N-morpholino)-2-hydroxypropanesulfonic sulfonic acid (MOPSO), 4-morpholinepropanesulfonic aicd (MOPS), 4-(2-hydroxyethyl)piperazine-1-ethanesulfonic acid (HEPES), phosphate, citrate, ammonium, acetate, and the like, can be used as long as they buffer at the desired pH.

The starting load material comprising the rSAg is exposed to an HIC matrix under conditions that promote binding of endotoxin to the HIC matrix but do not substantially promote binding of the rSAg. For example, the conditions under which the HIC chromatography is conducted will typically promote binding of 50% or more of the endotoxin present in the starting load material to the HIC matrix. Often, 75-80% or more of the endotoxin in the starting load material will be retained by the matrix (i.e., it will not be present in a greater than 15-20% of the starting material endotoxin concentration in one or more flow through fractions (FTs) in which the majority (e.g., 60-75% or greater) of the rSAg is collected during the HIC purification step). In certain embodiments, 80-90% or more of the endotoxin in the starting load material will be retained by the matrix, while only 10-20% of the endotoxin present in the starting load material will be collected with the rSAg for further purification according to the methods herein. At the same time. the HIC matrix does not substantially promote binding of the rSAg. For example, the conditions under which the HIC chromatography is conducted will typically lead to binding of less than 40% of the total rSAg present in the starting load material to the HIC matrix. Often, less than 25-30% of the rSAg in the starting load material will be retained by the matrix (i.e., it will be present in a greater than 70-75% of the starting material SAg concentration in one or more flow through fractions (FTs) in which the majority (e.g., 60-75% or greater) of the rSAg is collected during the HIC purification step). In certain embodiments, as little as 10 to about 20% or less of the starting rSAg is retained on the HIC column, while as much as 80 to about 90% or more of the rSAg present in the starting load material is collected from the HIC step for further purification according to the methods herein.

HIC substrates, also referred to as matrices or resins, that are suitable for use within the present invention are typically alkyl- or aryl-substituted HIC matrices. In certain embodiments, butyl-, octyl-, or phenyl-substituted matrices are used. For example, various suitable HIC resins for purifying rSAgs and separating them from endotoxins have immobilized phenyl functional groups. Phenyl-based HIC substrates from different vendors exhibit different efficiency for resolving SAgs and endotoxins. Exemplary phenyl-HIC resins include PHENYL TOYOPEARL media (TosoHaas, Philadelphia, Pa.), PHENYL SEPHAROSE FAST FLOW (low and high substitution); and TSK PHENYL 5PW. Useful HIC functional groups include the alkoxy, butyl, and isoamyl moieties. Other HIC-immobilized functional groups can also function in purification of SAgs and separation of SAgs from endotoxins. For example octyl groups, such as those on OCTYL SEPHAROSE CL4B media from Pharmacia, and propyl groups, such as those on High Propyl media from Baker, are useful, as are alkoxy, butyl, and isoamyl functional group resins. Other exemplary HIC matrices for use within the invention comprise a butyl-substituted, polymethacrylate hydrophobic interaction chromatography matrix (e.g., TOYOPEARL BUTYL 650M or TOYOPEARL BUTYL 650C, TosoHaas, Philadelphia, Pa.).

HIC matrix supports useful in the practice of the present invention include synthetic polymers, e.g., polystyrene, poly(methacrylates), and the like, cellulose, dextrans, agarose, cross-linked agarose, and the like. Typically, the HIC matrix is in a column through which the starting load of rSAg in a suitable buffer is passed.

HIC is demonstrated herein to be particularly useful for purification of SAgs, and for separation of SAgs from endotoxins. Surprisingly, conventional HIC strategies aimed toward retaining a target compound of interest on the HIC matrix are not operable for high quality, high yield purification of rSAgs. On the contrary, the methods disclosed for the first time herein involve collection of rSAg in the flow through (FT), whereas the undesired endotoxin contaminant present in the starting load material is retained at a high level of separation, as described above, on the HIC matrix.

In certain embodiments, the use of HIC to purify SAgs in a FT from a starting load material derived from a recombinant expression system as described above (e.g., a crude bacterial cell lysate or resolubilized ammonium sulfate pellet obtained therefrom) yields a substantially pure, or even a homogeneous, SAg sample. By “substantially pure” is meant a degree of purity of total rSAg, e.g., rSEB as compared to total protein in a sample collected following HIC, where there is at least 70% SAg, commonly at least 80%, and often at least 90% or greater. In certain embodiments, the SAg preparation resulting from HIC of the starting load material is substantially homogeneous, i.e., 95%-99% or greater as a ratio of the total protein present in an HIC-collected sample.

In related embodiments, HIC applied to purify SAgs according to the methods herein yields a HIC-collected sample that is substantially free of endotoxin. By “substantially free of endotoxin” is meant a composition in which the concentration of endotoxin compared to total protein is less than about 10%, preferably less than about 5%, typically less than 2%. Often the resulting collected sample (e.g., pooled FTs) following HIC are “essentially free” of endotoxin, wherein the concentration of endotoxin compared to total protein is less than about 1%. Alternatively, the collected sample from HIC may be considered essentially free of endotoxin when the endotoxin levels in the sample (or a pharmaceutical formulation derived from the sample, e.g., by further purification and/or admixture with excipients, buffers, carriers, and the like) is below an acceptable level of endotoxin activity for an indicated clinical use. For example, certain clinical uses allow for endotoxin levels that are less than about 100 endotoxin units (EUs) per milliliter, while other uses may require safe administration levels of approximately 10 EUs, 1 EU or less. In certain embodiments of the invention, these levels are met by the HIC purification, whereas in others the purification yields levels of endotoxin below the level of detectability (approx. 0.1 EU/ml). In other aspects of the invention, the HIC purification product is substantially free of lipopolysaccharide (LPS) and/or DNA, wherein the product contains less than about 20%, often less than about 10% to about 15%, and as little as about 1% to about 5% or less of the LPS and/or DNA levels present in the starting load material.

Prior to contacting a solution of rSAg with the HIC matrix, the pH and/or salt concentration of the rSAg starting load material, typically suspended in a suitable buffer, can be adjusted. The pH is typically adjusted to between about 6.0 and about 8.0, often between about 6.5 and about 7.5, and in certain embodiments to a value of about 7.0. In exemplary embodiments, the salt concentration (e.g., ammonium sulfate) is adjusted to between 0.5 M and 2.0 M, often between about 0.75 M and about 1.5 M, and in certain embodiments to about 1.0 M. Other salts suitable for such use include sodium sulfate, potassium sulfate, ammonium sulfate, potassium phosphate, sodium acetate, ammonium acetate, sodium chloride, sodium citrate, and the like. The column, buffers, and load material are typically maintained between about 0° C. and about 15° C., often between about 1° C. and about 10° C., and in certain embodiments between about 2° C. and about 8° C. for the duration of the separation.

The quantity of HIC matrix used in the practice of this invention may vary. Typically, about 0.05 to about 1 liter of matrix per gram of rSAg is used. Typically, the rSAg-containing load material is contacted with the HIC matrix for at least about 1 to 30 minutes. Optionally, part, or all, of the flow through (FT) containing rSAg from the HIC separation can be subjected to a second round of HIC purification prior to the next step in the purification protocol.

In additional embodiments of the invention, an intermediate chromatography step following the HIC step is included which comprises a buffer exchange step. Briefly, this comprises a desalting procedure according to any of a wide variety of known methods. In one exemplary embodiment, the desalting follows directly or indirectly the HIC step, and employs a buffer exchange, e.g., using Sephadex G25 fine resin (APB). The resin is equilibrated with a suitable buffer and the HIC product is loaded (e.g., at about 25% column volume (CV)) at a linear flow rate (e.g., of about 30 cm/hour). The load is then washed (e.g., using 1 CV of 25 mM sodium phosphate, pH 6.0). These column runs may be repeated as required to adequately desalt the SAg-containing material in preparation for subsequent cation exchange separation.

In yet additional embodiments, directly or indirectly following the desalting step, the SAg-containing material is subjected to cation exchange chromatography. This involves contacting an SAg-containing sample solution with a suitable quantity of a cation exchange matrix under conditions allowing adsorption to the matrix of at least about 60%, more typically at least about 75%, and in certain embodiments 95% or more of the total SAg from the solution. The SAg-containing solution may be contacted with the cation exchange matrix in various ways. For example, the cation exchange matrix may be in a vessel into which the SAg-containing medium is introduced, followed by decanting of the SAg-depleted medium from the cation exchange matrix. Alternatively, the cation exchange matrix can be added to the vessel containing the SAg-containing medium, followed by removal of SAg-depleted solution from cation exchange matrix. Typically, the cation exchange matrix is in a column through which the SAg-containing medium is allowed to flow.

Matrices that can be used in the cation exchange step are well known in the art. Typically, the cation exchange matrix is compatible with a high flow rate, i.e., able to withstand high pressures, possesses a macroporous structure, and is capable of binding SAg over a wide pH range. Matrix supports such as cellulose, polystyrene, dextrans, agarose, cross-linked agarose, and the like, can be used for the cation exchange matrix. Exemplary cation exchange matrices for use in this step are sulfylpropylated (“SP”) matrices, for example, TOYOPEARL SP550C (TosoHaas, Philadelphia, Pa.). In certain embodiments, SP-SEPHAROSE High Performance (Pharmacia; highly cross-linked 6% agarose; particle size of 34 microns), MACROPREP HIGH S Cation-exchange (BIO-RAD Laboratories; strong cation exchange; SO₃⁻ functional group; nominal particle size of 50 μm; nominal pore size of 1000 Å), polyaspartic acid resin (e.g., PolyCAT A); Silica gel (underivatized); Phenyl Sepharose Fast Flow Low Substitution (Pharmacia; highly cross-linked 6% agarose; particle size of 45-165 microns); PHENYL TOYOPEARL 650 M (TosoHaas; particle size of 40-90 microns); or Fractogel EMD SO₃⁻650 S (EM Separations, a U.S. associate of E. Merck (Germany); particle size of 25-40 μm) can be used.

Buffers for the cation exchange aspect of this invention generally have a pH in the range of about 5 to about 8, and in certain embodiments about pH 6. Buffers that will control the pH within this range include, for example, citrate, succinate, phosphate, morpholinoethane sulfonic acid (MES), ADA, 2-[bis(2-hydroxyethyl)amino]-2-(hydroxymethyl)-1,3-propanediol (31S-TRIS) Propane, piperazine-N—N′-bis(ethane sulfonic acid) (PIPES), ACES, imidazole, diethylmalonic acid, MOPS, MOPSO, N-Tris(hydroxymethyl) methyl-2-aminoethane sulfonic acid (TES), TRIS buffers such as TRIS-HCl, HEPES, N-(2-hydroxyethyl)-piperazine-N′-2-ethane sulfonic aicd (HEPPS), N-Tris(hydroxymethyl)methyl glycine (TRICINE), glycine amide, BICINE, glycylglycine, and borate buffers. An exemplary buffer used herein for cation exchange is sodium phosphate.

One of ordinary skill in the art is credited with recognizing that prior to contacting the SAg-containing sample solution with the cation exchange matrix, the matrix may need to be regenerated and/or activated. Regeneration and/or activation should be carried out according to the recommendations of the matrix vendor and methods known in the art.

The quantity of cation exchange matrix material used is typically at least about 0.031, to 1.0 liter of matrix per gram of SAg. Typically the SAg-containing solution is contacted with the cation exchange matrix for about 0.1 to 1 hour, or longer. Once the SAg-containing solution has been contacted with the cation exchange matrix to allow SAg adsorption, the SAg-depleted solution is removed. This can be done in various ways, e.g., filtration, decantation, centrifugation, and the like. In a particular embodiment, contacting of the SAg-containing solution and removal of the SAg-depleted medium are done in one step by passing the SAg-containing medium through a chromatography column comprising the cation exchange matrix.

Once the SAg has been adsorbed onto the cation exchange matrix, and prior to SAg elution, it may be desired to wash the SAg-loaded matrix, for example with about 1 to about 10 column volumes of a suitable buffer or dilute, weak acid. Exemplary dilute, weak acid solutions include acetic acid or phosphoric acid solutions, at a concentration of about 0.02 M. This additional step can be useful to remove impurities, including multimeric and/or misfolded SAg forms, that are bound less tightly to the cation exchange matrix than naturally conformed, monomeric SAg. In the present invention, such high ionic strength washing steps can be avoided in the cationic exchange step, because it may be advantageous to recover multimeric or misfolded monomeric forms of SAg, which can be converted to a natural, monomeric state by subsequent unfolding/refolding steps known to those skilled in the art, thereby increasing overall yield of biologically active SAg.

After the cation exchange matrix has been loaded and/or washed (as described above), the SAg is eluted by contacting the matrix with a sufficient quantity of a solvent system (“elution buffer”) which has a sufficiently high pH or ionic strength to displace substantially all of the SAg from the matrix. The elution buffer should comprise a buffering agent suitable for maintaining a pH in the range of about 5.0 to about 7.0, typically about 6.0, and a salt concentration of about 0.2 M to about 1.0 M, typically about 0.5 M. An exemplary elution buffer comprises 0.5 M NaCl. Alternative elution buffers include sodium acetate, sodium phosphate, and sodium chloride, among others known in the art. The quantity of elution buffer used is variable but is preferably between about 1 and about 10 column volumes (CV). In one exemplary elution protocol employed herein, the cation exchange matrix was exposed to a 10 CV linear gradient from 0 to 50% sodium phosphate, pH 6.0, and 0.5 M NaCl. Multiple fractions proximal to the start of the peak can be collected separately, and peak material above 0.25 absorbance Units (AU) can be collected in a single fraction. These fractions can be pooled subsequently based on purity analyses and other considerations.

Following or preceding the cation exchange step, anion exchange chromatography can be employed as an optional step in the rSAg purification process. This was evaluated herein following POROS® 20S chromatography for ensuring endotoxin and DNA removal. Although there was no detectable impact on yield (rSEB was determined not to bind significantly), there was no apparent gain in purity or activity. Nonetheless, anion exchange chromatography may prove useful at larger production scales to add robustness to the system for the removal of residual endotoxin and DNA.

In additional aspects of the invention, rSAg product obtained following HIC, buffer exchange, and cation exchange chromatography can be further refined and concentrated prior to additional, optional steps of final polishing and formulation. For example, an exemplary further purification step using POROS® 20HS media is described below. By this procedure, small amounts of dimer and lower-molecular-weight contaminants were separated from the main SAg product peak. The product from this final step constitutes an intermediate bulk product and has been found to be extremely stable for over 12 months at −70° C. Consistent with FDA guidelines and requirements for current good manufacturing practices (CGMP), the stability of the rSAg product in this buffer formulation allows the extended storage of a highly purified bulk product ready for final polishing and formulation (Manning et al., Pharm. Res. 6:903-918, 1989; and Wang, W., Int. J. Pharm. 185:129-188, 1999, each incorporated herein by reference).

In alternate embodiments of the invention combining the above described steps of HIC, buffer exchange, and cation exchange chromatography, and optional further refinement and purification, SAgs are purified from the starting load material (e.g. crude bacterial cell lysate or resolubilized ammonium sulfate pellet) to a final form that is substantially pure, or homogeneous. Thus, the final purity of the rSAg product, e.g., rSEB, as a percentage of total protein therein is at least 70% SAg, commonly at least 80%, and often at least 90% or greater. In certain embodiments, the SAg preparation steps of HIC, buffer exchange, and cation exchange chromatography render the final product substantially homogeneous, i.e., about 95% to about 99% or greater as a ratio of the total protein therein.

In related embodiments, the combined steps of HIC, buffer exchange, and cation exchange chromatography yield a final product that is substantially free of endotoxin. Thus, the final product has a concentration of endotoxin compared to total protein that is less than about 10%, typically less than about 5%, more typically less than 2%. Often the resulting final product is “essentially free” of endotoxin, as described above, wherein the concentration of endotoxin compared to total protein is less than about 1%, or wherein the endotoxin levels are less than about 100 endotoxin units (EUs), often less than approximately 10 EUs, 1 EU or even below the level of detectability (approx. 0.1 EU/ml). In related aspects of the invention, the combined steps of HIC, buffer exchange, and cation exchange chromatography yield a final purification product that is substantially free of lipopolysaccharide (LPS) and/or DNA, wherein the product contains less than about 20%, often less than 10% to about 15%, and as little as about 1% to about 5% or less of the LPS and/or DNA levels present in the starting load material.

The final SAg purification product of the invention typically exhibits clinically acceptable safety characteristics, e.g., as determined by gram-negative bacterial endotoxin levels, kinetic limulus amebocyte lysate assay (BioWhittaker, Walkersville, Md.), ELISA or other immunoassays for host cell proteins, DNA Threshold System assay (Molecular Devices, Sunnyvale, Calif.), or other methods. In certain embodiments, the final purified rSAg (e.g., rSEB) product meets all of the physiochemical, safety, and potency attributes required for a Phase I clinical candidate (U.S. Code of Federal Regulations, Title 21, Parts 210, 211 and 600, The National Archives and Records Administration Office of the Federal Register, Government Printing Office, Washington, D.C., 1998). In exemplary embodiments, matrix-assisted laser desorption/ionization-time-of-flight (MALDI-TOF) analysis demonstrates a single major species with a molecular weight of 28.3 kDa consistent with the predicted size of rSEB. Only a single N-terminal amino acid sequence corresponding to the cDNA predicted sequence was identified. SDS-PAGE (reduced and non-reduced), reverse phase HPLC on C18 resin, and capillary zone electrophoresis (CZE) demonstrated that a rSEB protein was purified to homogeneity. Analytical SEC (size exclusion chromatography) showed this product to be monomeric in solution. Capillary IEF showed no evidence of proteolytic degradation or aggregation. No changes in any of these parameters were observed for the rSEB product after approximately 12 months in storage at −70° C. In addition to these safety characteristics for rSEB, no E. coli host cell proteins were detected using a validated ELISA assay (LOD=5 ng/mg). Endotoxin and total genomic DNA were below the level of detection (0.06 EU/mg and 15.6 pg/mg, respectively). When tested at a concentration of 1.04 mg/ml, the rSEB final product passed both the USP rabbit pyrogen test and general safety assessment.

The rSAgs produced by the methods of the invention are useful as safe, effective reagents for administration to mammals, including humans, for example as a biodefense vaccine or for the treatment of sepsis or toxic shock. In this regard, the potency of an exemplary rSAg (rSEB) is demonstrated herein using an art accepted mammalian (murine) model system. When immunized with 5 μg of rSEB, 70% of mice challenged with wild-type SEB were protected. 100% of mice were protected when challenged with wild-type SEB following immunization with 20 μg of the isolated recombinant protein.

Compositions obtained by the purification processes described herein will comprise substantially pure SAg. Often the compositions will comprise essentially pure or homogeneous SAg, and will be substantially free of one or more contaminants selected from endotoxin, LPS, and DNA. The purity of SAg in the compositions of the invention can at times be greater than 99.5%.

Purified rSAgs according to the invention are suitably formulated into a composition with a carrier, typically a pharmaceutical composition with a physiologically acceptable carrier. These pharmaceutical compositions are preferably in a sterile, stable, liquid or lyophilizable form, and comprise the rSAg in amounts suitable for effective, safe pharmaceutical administration, particularly as a vaccine.

Generally, the formulations are prepared by contacting the purified rSAg uniformly and intimately with liquid carriers or finely divided solid carriers or both. Then, if necessary, the product is shaped into the desired formulation. Typically, the carrier is a parenteral carrier, more commonly a solution that is isotonic with the blood of the recipient. Examples of such carrier vehicles include water, saline, Ringer's solution, dextrose solution, and the like. Non-aqueous vehicles such as fixed oils and ethyl oleate are also useful herein, as well as liposomes, and the like.

The carrier suitably contains minor amounts of additives such as substances that enhance isotonicity and chemical stability. Such materials are non-toxic to recipients at the dosages and concentrations employed, and can include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, trehalose, glucose, mannose, or dextrins; chelating agents such as EDTA; sugar alcohols such as mannitol or sorbitol; counter-ions such as sodium; and/or nonionic surfactants such as polysorbates, poloxamers, or PEG. The final preparation can be a liquid or lylphilized solid. In injectable, e.g., vaccine, formulations, the rSAg can be combined with an effective adjuvant to enhance the recipient's immune response. Many useful adjuvants are known for this purpose, including but not limited to, aluminum hydroxide (e.g., ALHYDROGEL).

Administrable SAg compositions generally are placed into a container having a sterile access port, for example, an intravenous solution bag or vial having a stopper pierceable by a hypodermic injection needle. The above formulations are also suitable for in vitro uses. The SAg formulation can be stored in unit or multi-dose containers, for example, sealed ampoules or vials, as an aqueous solution, or as a lyophilized formulation for reconstitution. As an example of a lyophilized formulation, 10 ml vials are filled with 5 ml of sterile-filtered 1% (w/v) aqueous SAg solution, and the resulting mixture is lyophilized. The infusion solution is prepared by reconstituting the lyophilized rSAg using bacteriostatic Water-for-Injection.

A therapeutically effective dose of an SAg formulation can be administered to a recipient to induce an immune response to enterotoxin so as to reduce or eliminate symptoms of a disease or condition mediated by a bacterial SAg. By “therapeutically effective dose” herein is meant a dose that produce an immune response sufficient to reduce or eliminate the adverse effects for which it is administered. For example, boosting antibody titers for patients at risk for toxic shock syndrome and the other disorders of common etiology. The exact dose will depend on the disorder to be treated, and will be ascertainable by one skilled in the art using known techniques. In general, the SAg formulations of the present invention are administered at about 0.01 μg/kg to about 10 mg/kg per day. Preferably, from about 0.1 to about 0.3 μg/kg. In addition, as is known in the art, adjustments for age as well as the body weight, general health, sex, diet, time of administration, drug interaction and the severity of the disease may be necessary, and will be ascertainable with routine experimentation by those skilled in the art. Typically, the clinician will administer SAg formulations of the invention until a dosage is reached that confers the desired response, e.g., protective or increased immunity in a recipient.

The following examples are offered by way of illustration and not by way of limitation. The disclosures of all publications cited in this specification are incorporated herein by reference for all purposes.

### EXAMPLE I

### Generation of the rSEB Clone and Construction of Master Cell Bank for Production of rSEB

Site-specific mutagenesis was performed using gene templates isolated from a clinical isolate of a Staphylococcus aureus strain (14458) that expressed SEB (sequence derivation of GenBank accession M11118). The gene was originally inserted into pBluescript II KS(+) by a PCR-based cloning strategy. Helper M13-phage were used to rescue single-stranded DNA template that was propagated in a dut,/ung mutant strain of E. coli (CJ236). Modified T7 polymerase (Sequenase, U.S. Biochemical Corp., Cleveland, Ohio) was used to synthesize second-strand DNA from synthetic oligonucleotides harboring the altered codon and single-stranded, uracil-enriched M13 templates. Mutagenized sequences were confirmed by DNA sequencing using synthetic primers, derived from known sequences or universal primers. Replicative form, double-strand DNA was isolated form E. coli host cells and the insert was shuttled to pSE380, for initial expression studies, and finally to pET24b for scale-up production by first introducing a NdeI site into the 5′ end of the gene by PCR. An original seed stock of E. coli containing the rSEB gene ((899445C) on plasmid pET 24b was used to prepare a master cell bank (MCB).

Two 1-L, triple-baffled shake flasks were batched with 24 g/L of yeast extract, 12 g/L of soytone, 4 g/L of glycerol, 2.31 g/L of KH₂PO₄, 12.5 g/L of K₂HPO₄, and 50 μg/ml of kanamycin, and inoculated with single-colony isolates grown on TSA plates. The flasks were placed on a rotary shaker at 200 rpm and incubated overnight at 37° C. to an OD₆₀₀ of approximately 14. The flasks were then pooled, and glycerol was added to a final concentration of 13%. The cells were aliquoted at 1 ml in a 2-ml cyrovial and subjected to a slow-rate freeze. Vials were tested for purity, Gram stain, biological type, and rSEB gene-insert sequence integrity. The average cell count was 3.98×10⁸ CFU/ml.

### EXAMPLE II

### Fermentation for Production of rSEB

The build up of seed stock was conducted in two stages. In the first stage, three 500-ml, triple-baffled, shake flasks were batched with 100 ml of sterile seed medium (24 g/L yeast extract, 12 g/L soytone, 4 g/L glycerol, 2.31 g/L KH₂PO₄, 12.5 g/L K₂HPO₄, and 50 μg/mL kanamycin). Each first-stage seed flask was inoculated aseptically with 1 ml (1% v/v) of pooled material from four thawed vials of the MCB. First-stage seed flasks were placed on a rotary shaker at 250 rpm and incubated at 37° C. One of the flasks was selected to obtain time-course samples at one-hour intervals. The time-course samples were processed for OD₆₀₀, pH, TSAG plate, and observed by wet mount. Based on the growth curve (OD₆₀₀ values vs time), the first-stage seed culture was considered ready to scale to the second-stage seed when late log phase growth was observed. In the second stage, three 2000-ml, tripled baffled, shake flasks were batched with 480 ml of the sterile seed medium formulation described above. Each second-stage seed flask was inoculated aseptically with 9.6 ml (2% v/v) of the first-stage seed culture. All second-stage seed flasks were placed on a rotary shaker at 250 rpm and were incubated at 37° C. One of the flasks was selected to obtain time-course samples at one-hour intervals. The time-course samples were processed for OD₆₀₀, pH, TSAG plate, and observed by wet mount. Criteria to scale the second-stage seed to the production fermenter were based on the resulting growth curve (late log phase or early stationary phase) and a requirement to reach a specified OD₆₀₀ (8.0±2.0). The production stage was conducted in an 80-L fermenter with a working volume of 48 L. The production fermenter was batched with 24 g/L of yeast extract (DIFCO, Detroit Mich.), 12 g/L of soytone (DIFCO), 4 g/L of glycerol (Fisher, Pittsburgh, Pa.), 2.31 g/L of KH₂PO₄ (Fisher), 12.5 g/L of K₂HPO₄ (Fisher), 0.1% v/v of P2000 antifoam, and 50 μg/ml kanamycin (Sigma Chemical Company, St. Louis, Mo.). The fermenter was maintained at a temperature of 37° C., an agitation rate of 177 to 461 rpm, an aeration rate of 16 to 58 slpm, and a vessel pressure of 3 to 7 psig. The % DO within the fermenter was maintained at >20% by first adjusting the sparging and then the agitation rate incrementally. Time-course samples were obtained aseptically from the fermentation vessel at least at one-hour intervals and processed for OD₆₀₀, pH, glutamate, TSAG plate, and wet mount. The culture was aseptically fed 2.4 L of a sterile 10× nutrient feed (24 g/L yeast extract, 12 g/L soytone, 4 g/L glycerol, 9.6 L purified water) at a rate of 50 mL/minute when the OD₆₀₀ reached 8±2, or the amount of glutamate in the production culture was less than 50% of the original glutamate concentration at inoculation. This 10× nutrient feed was continued as long as the DO levels were greater than 20%. The culture was induced with filter-sterilized, dioxane-free IPTG at a 1 mM final concentration when the OD₆₀₀ reached 12.5±2.5. Immediately after IPTG induction, the culture was fed with 2.4 L of a filter-sterilized 20× yeast nitrogen base solution (2 g/L yeast nitrogen base, 2.4 L purified water). The culture was prepared for harvest 4 h after IPTG induction. Prior to harvest, a final sample was obtained aseptically and processed for TSAG plate, OD₆₀₀, pH, glutamate, and DNA sequencing. The sample was also observed by wet mount. In preparation for harvest, the fermenter was chilled to 10° C., the agitation rate reduced to 55 rpm, and the aeration rate adjusted to 16 slpm.

### EXAMPLE III

### Cell Recovery, Disruption, and Pre-Column Treatment for Production of rSEB

Each step of the recovery process was performed using equipment cooled with chilled water (<55° F.). The fermented broth was transferred to a chilled tank and concentrated using a Microgon 0.2 μm, 3 square meter surface area, hollow-fiber module (Spectrum Laboratories, Rancho Dominguez, Calif.). The broth was recirculated through the module using a peristaltic pump. The filtrate flow from the hollow fiber module was directed to a suitable waste container. The broth was concentrated from a starting volume of 55 L to a calculated cell density of approximately 250 to 300 g/L. Residual soluble medium components were removed from the retentate by diafiltration with 6 volumes of 50 mM phosphate, pH 7.4, and 100 mM sodium chloride. At the completion of the diafiltration, the filter was rinsed with a calculated volume of 50 mM phosphate, pH 7.4, 100 mM sodium chloride, and 140 mM EDTA to bring the final EDTA concentration up to 20 mM.

Cells were disrupted at 55 MPa (8,000±500 psig) by passing the cell suspension twice through a model 15MR8TA Gaulin high-pressure homogenizer equipped with a heat exchanger for cooling (APV Gaulin, Inc., Everett, Mass.). Disruption efficiency after two passes was >90% as determined by A₆₀₀. The cell lysate was clarified by diafiltration with 6 volumes of 50 mM phosphate, pH 7.4, 100 mM sodium chloride, and 20 mM EDTA using a 0.1 μm, one-square meter Septoport filter module (NC-SRT, Inc., Carey, N.C.). The retentate was recirculated by means of a rotary lobe pump. The lysate was kept in a chilled vessel during this operation, and the filtrate was collected in chilled, sterile, depyrogenated carboys.

The lysate filtrate was then subjected to tangential flow filtration (TFF) using an Amicon S10Y100, 100-kDa spiral membrane module with a 1 square meter surface area (Millipore, Bedford, Mass.). The retentate was recirculated through the module using a peristaltic pump. The filtrate was fed directly from the module into a filtrate receiver tank that was jacketed with chilled water. The retentate was concentrated to 1/30-1/50 of the starting volume. The 100-kDa filtrate was then concentrated using two Amicon SI0Y30 30-kDa spiral membrane modules, 2 square meters surface area, in tandem (Millipore). The retentate was recirculated through the module using a peristaltic pump. The retentate vessel remained chilled during this procedure. The retentate was first concentrated to 1/10-1/20 of the starting volume, then diafiltered with 10 volumes of 50 mM phosphate, pH 7.4, 100 mM sodium chloride, and 20 mM EDTA.

The 30-kDa retentate was subjected to differential ammonium sulfate precipitation by adding solid ammonium sulfate to 45% saturation. The pH was monitored during this procedure and maintained between 7.0 and 7.4 by adding 0.5 M ammonium hydroxide. The suspension was then stored for 4 h minimum at 2 to 8° C., after which the precipitate was recovered by means of bottle centrifugation at 13,700×g for 15 min in a 2° C. to 8° C. refrigerated centrifuge Beckman, Palo Alto, Calif.). The supernatant was decanted into a sterile, depyrogenated container, and the pellets were discarded. Solid ammonium sulfate was then added to the 45% precipitation supernatant to a final concentration of 75% saturation. The pH was monitored during this procedure and maintained between 7.0 and 7.4 by adding 0.5 M ammonium hydroxide. The suspension was stored for 4 h minimum at 2° C. to 8° C., after which the precipitate was recovered by means of bottle centrifugation (Beckman) at 13,700×g for 15 min in a 2° C. to 8° C. refrigerated centrifuge (Beckman). The supernatant was decanted as waste, and the recovered pellets were stored at or below −60° C. in 250-ml polycarbonate screw-cap containers.

One hundred and six grams of frozen ammonium sulfate pellets were combined into a single, sterile, pyrogen-free container and solubilized by the addition of 200 ml (2 ml/g) of cold 25 mM sodium phosphate buffer, pH 7.0. Pellets were completely solubilized by gentle stirring. Samples were removed for determination of protein content by BCA assay. Based on the protein concentration, solubilized pellets were diluted by the addition of 1.0 M ammonium sulfate, 25 mM sodium phosphate buffer, pH 7.0, to obtain a final protein concentration of 1.5 mg/ml. Solubilized pellets were then filtered into a sterile, chilled, pyrogen-free sterile vessel using a 0.22 μm Millipack filter (Millipore). In-process testing consisted of SDS-PAGE, protein concentration, endotoxin, and bioburden determination.

Briefly summarizing the results of the foregoing Examples, analysis of the shake-flask cultures by SDS-PAGE and Western blot analysis showed a band at approximately 30 kDa that was not evident prior to induction. Media optimization studies showed that, for high-density fermentations, yeast extract-based media performed very well. Addition of glucose was found to suppress the final OD₆₀₀ and was omitted from the final media formulation. Finally, addition of trace elements (NH₄SO₄, ZnSO₄.7H₂O, CuSO⁴.5H₂O, MnSO⁴.H₂O, FeCl³.6H₂O, CoCl².6H₂O, Na₂MoO⁴.2H₂O) were found to substantially improve the final yield. The final media formulation, containing soytone, yeast extract, phosphate buffer, and glycerol, was selected for further evaluation.

When cultured at 37° C. and induced with 1 mM IPTG, approximately 70% of the protein was found in the soluble fraction and 30% in the insoluble fraction as inclusion bodies. In order to drive more of the product into the soluble fraction, induction conditions were evaluated at 32° C. and 37° C. using 0.1, 0.5, or 1 mM IPTG (Friehs and Reardon, Adv. Biochem. Eng. Biotechnol. 48:55-74, 1993; Hannig and Makrides, Trends Biotechnol. 16:54-60, 1998; Moore et al., Protein Express. Purif. 4:160-163, 1993; Studier and Moffatt, J. Mol. Biol. 189:13-130, 1986; Makrides, Microbiol. Rev. 60:512-538, 1996, each incorporated herein by reference). The results of a 20 liter pilot fermentation showed that >90% of the product could be expressed as a soluble protein when the culture was induced at an OD₆₀₀ of about 10 to about 15 with 1 mM IPTG at 37° C. for a total of 4 h. The final OD₆₀₀ at the 20-liter scale was approximately 40.

In order to avoid the potential safety concerns associated with using a nitrogen source derived from bovine or other animal products, peptones from non-animal sources were evaluated for their ability to support growth (Table 1). The results showed that although tryptone was superior to the other nitrogen sources evaluated, soytone also was judged to be satisfactory for supporting large-scale fermentation.

TABLE 1
Evaluation of Nitrogen Sources Derived from Non-Animals
for the Fermentation of E. coli BL21/SE

| Media Composition | OD₆₀₀ (12 h) |
|---|---|
| Yeast Extract + Tryptone | 9.07 |
| Yeast Extract + Soytone | 8.48 |
| Yeast Extract + #3 Phytone | 6.21 |
| Yeast Extract + Phytone | 7.53 |

Based on these results, further optimization efforts focused on 1) batch vs. fed-batch fermentation, and 2) nutritional feeding during the fermentation. Twenty-liter pilot scale studies showed that the final biomass yield was poor in the absence of feeding (Table 2). Additional carbon-source feeding did not enhance the final biomass yields. However, the highest cell densities were achieved by feeding with a combination of yeast extract, soytone and glycerol. The average fermentation period for all 20-liter pilot runs was 10 to 12 h. Expression levels were observed to be consistent at approximately 10% of the total soluble protein as judged by densitometric analysis of reducing SDS-PAGE gels.

TABLE 2
Influence of Feeding on Fermenter Final Biomass

| | | | | Cell Mass |
|---|---|---|---|---|
| Feed | Start OD | Final OD | Induction Time (h) | (g) |
| None | 3 | 9 | 3 | 123 |
| S + YNB | 13 | 18 | 4 | 250 |
| Y + YNB | 12.3 | 28 | 4 | 400 |
| G + YNB | 10.2 | 13.3 | 4 | 200 |
| SYG + YNB | 12.5 | 46.7 | 4 | 800 |
| SYG + YNB | 13.9 | 41 | 4 | 900 |

SYG: soytone-yeast extract-glycerol
YNB: yeast nitrogen base

Final volumes in the SYG & YNB pilot fermentations were not identical due to different sampling rates employed. There runs, however, demonstrate the ability to achieve a final OD ₆₀₀ 240 and an achievable cell mass yield required of 900 g

### EXAMPLE IV

### Column Chromatography for Production of rSEB

Column 1. In an initial column chromatography step, a 5.0 cm (i.d.) XK chromatography column (Amersham Pharmacia Biotech (APB), Uppsala, Sweden) was packed with 650 ml of depyrogenated phenyl-sepharose, fast-flow, high-substitution HIC resin (APB). In development studies, the resin load limit was determined to be approximately 20 mg protein per ml of resin at 2° C. to 8° C. The separation was performed by first equilibrating the resin with 10 column volumes (CV) of buffer (25 mM sodium phosphate, pH 7.0, 1.0 M ammonium sulfate) at 60 cm/hour. Resuspended ammonium sulfate pellets were loaded onto the column at 60 cm/hour. The column, buffers, and load material were maintained at 2° C. to 8° C. for the duration of the separation. The column was washed with 2.0 CV of equilibration buffer at 60 cm/hour. The flow through (FT) with a UV absorbency (OD₂₈₀ nm)>0.1 AU above baseline was collected into a sterile, pyrogen-free container until the UV absorbency returned to baseline.

Column 2. A second chromatography step was employed comprising a buffer exchange using Sephadex G25 fine resin (APB). A 20.0-cm (i.d.) BPG column (APB) containing 12.2 L of resin was equilibrated with 5 CV of buffer (24 mM sodium phosphate, pH 6.0) at 30 cm/hour prior to loading. Product was loaded at 25% CV at a linear flow rate of 30 cm/hour. The load was washed using 1 CV of 25 mM sodium phosphate, pH 6.0. Three column runs were required to desalt all the material in preparation for the cation exchange separation.

Column 3. A third chromatography step was employed comprising a cation exchange on POROS® 50HS media (PerSeptive Biosystems, Framingham, Mass.). In development studies, the resin load limit was determined to be approximately 10.0 mg protein/ml of resin. The desalted HIC-purified material was loaded onto a 9-cm (i.d.) Vantage A2 column (Millipore) containing 600 ml of POROS® 50HS media. The column was equilibrated with 25 mM of sodium phosphate, pH 6.0. The pooled peaks from the desalting column were loaded onto the cation-exchange column at a linear flow rate of 56 cm/hour. The column was washed with 5 CV of the equilibration buffer. Protein was eluted with a 10-CV linear gradient from 0 to 50% sodium phosphate, pH 6.0, and 0.5 M NaCl. Three fractions at the start of the peak were collected separately, and the peak material above 0.25 AU was collected as a single fraction. Fractions also were collected for the eluate below 0.25 AU on the trailing edge of the peak. Fractions were analyzed by SDS-PAGE and only those containing a single band of rSEB were pooled.

Column 4. A fourth chromatography step was employed comprising a desalting of the POROS® 50HS pool on Sephadex G25 fine media. The POROS® 50 pool was loaded onto a 20-cm (i.d.) column containing 12.8 L of media equilibrated in 25 mM sodium phosphate buffer, pH 6.0. A 20%-CV load was applied at a linear flow rate of 30 cm/hour. One CV of buffer was used to wash material after loading. All material with UV absorbency above baseline was collected into a pre-cooled container.

Column 5. A fifth chromatography step was employed comprising a cation exchange on POROS® 20HS media (PerSeptive Biosystems). The desalted rSEB pool was loaded onto a 9-cm (i.d.) Vantage A2 column containing 760 ml of POROS® 20HS media equilibrated in 25 mM sodium phosphate buffer, pH 6.0. The column was loaded at a linear flow rate of 56 cm/hour. Following the load, the column was washed with 5 CV of equilibration buffer 25 mM sodium phosphate buffer, pH 6.0. Proteins were eluted with a 10-CV linear gradient from 0 to 50% (25 mM sodium phosphate, pH 6.0), and 0.5 M NaCl. Three fractions were collected at the beginning of the peak elution (AU above 0.25) and continuing until the absorbence was below 0.25 AU. Fractions were analyzed by SDS-PAGE and only those fractions containing a single band of rSEB were pooled. The purified intermediate bulk product was sterile filtered using a 0.22 μm Millipak 100-unit (Millipore), aliquoted into sterile Nalgene PETG bottles, and frozen at −70° C.

Column 6. Purified intermediate bulk product underwent a final size exclusion chromatography SEC step immediately prior to dilution and vialing. A 5%-CV load of purified rSEB was injected onto a 5-cm (i.d.) XK column (APB) packed with 1700 ml of Superdex 75 prep-grade resin (APB) using a superloop device. The separation was accomplished at a linear flow rate of 30 cm/hour. Peak fractions were collected, tested by HP-SEC and pooled. Pooled fractions were then diluted in 50 mM glycine, pH 8.5, and 140 mM NaCl to a final target concentration of 80 μg/ml. Diluted product was filtered through a 0.22 μm Millipore Millipak 20-unit filter and stored at 4° C. for less than 24 h prior to vialing.

Fill and Finish. The 0.22 μm-filtered final recombinant protein product was filled into cleaned, sterile, depyrogenated 5-ml glass vials (West Company, Lionville, Pa.) with 13-mm butyl rubber stoppers (Wheaton, Millville, N.J.) and a flip-off, crimp-type seal (West Company). Vials were stored refrigerated at 2° C. to 8° C. for no longer than 48 h prior to controlled-rate freezing. Final product storage was at or below −70° C.

### EXAMPLE V

### Analytical Testing of rSEB

Total protein analysis. Total protein concentration of samples was determined using the BCA protein assay kit (Pierce Chemical Co., Rockford, Ill.) according to the manufacturer's instructions and using bovine serum albumin (Pierce) as the reference standard.

SDS-PAGE and Western blot analysis. Samples taken at various stages of the purification following Examples I-IV, above, were analyzed by SDS-PAGE using 4-20% gradient acrylamide gels (Novex, San Diego, Calif.), based on the method described by Laemmli (Nature 227:680-685, 1970, incorporated herein by reference). Samples were mixed with Tris-glycine SDS sample buffer containing β-mercaptoethanol as a reducing agent. Gels were stained with either Coomassie Brilliant Blue (Sigma Chemical Co., St. Louis, Mo.) or silver (APB). For Western blot analysis, proteins were transferred to a PVDF membrane (Novex) at 0.5 mA constant current in transfer buffer (25 mM Tris, 192 mM glycine, 20% methanol) for 1.5 h on ice. Blots were incubated in blocking buffer (PBS with 5% nonfat dry milk and 0.05% Tween® 20) for 1 h at 4° C. and then treated with the primary antibody, a mouse monoclonal antibody to rSEB for an additional hour at 4° C. Blots were washed three times for 10 min in blocking buffer and then incubated for 1 h at 4° C. with a goat anti-mouse IgG conjugated to alkaline phosphatase (AP) (Kirkegaard & Perry Laboratories, Gaithersburg, Md.) diluted 1/5000 in blocking buffer. Immunoreactive proteins were visualized by incubation with NBT-BCIP (Life Technologies, Rockville, Md.) substrate for 5 min at ambient temperature.

Reversed phase-HPLC. Samples were analyzed by reverse-phase high-performance liquid chromatography. 25-μg samples were injected onto a 2.1 mm×150 mm C18 column (Waters, Milford, Mass.). Protein was eluted from the C18 column with a gradient from 15% buffer A (0.1% TFA in ddH₂O) to 65% buffer B (99.9% Acetonitrile, 0.1% TFA). The separation was performed at ambient temperature, 1.0 ml/min, and was monitored at a wavelength of 210 nm. The separation was controlled and monitored with a Hewlett-Packard 1090 liquid chromatography workstation. Chromatograms were integrated and the area under the rSEB peak was reported as a percentage of the total area detected.

SEC-HPLC. Size exclusion chromatography was performed in phosphate buffered saline, pH 7.4, with 0.5 M NaCl using a G3000SW_{XL} (7.8 mm×30 cm, 5 μm particle size) column (TosoHaas, Montgomeryville, Pa.). The flow rate for the separation was 0.5 ml/min for a 30 min run. Sample application was 25 μl at 1 mg/ml. The separation was run on a Waters HPLC controlled with Millenium® software, with detection at a wavelength of 280 nm. rSEB purity was calculated as the area under the rSEB peak as a percentage of the total peak area detected.

Capillary zonal electrophoresis & capillary isoelectric focusing. rSEB was analyzed by Capillary Zonal Electrophoresis (CZE) at 22° C. using a Beckman P/ACE™ 5510 instrument equipped with a 37 cm long fused-silica capillary with a 50 μm i.d. The capillary was coated with 10% polyacrylamide for elimination of electroosmotic flow and adsorption of protein on the capillary wall. The running buffer was 100 mM acetic acid adjusted to pH 4.5 with TEA. The running voltage was 12 kV and the current generated was 24 μA. The detection wavelength was 200 nm. Sample buffer was diluted to reduce the salt concentration. High salt concentration was shown to split the protein peak. Under these conditions rSEB gives a single sharp peak at a migration time of about 10 minutes. rSEB was analyzed by capillary isoelectric focusing at 2° C. using a Beckman P/ACE™ 5510 instrument equipped with a 27 cm Beckman N—CHO capillary with a 50 μm i.d. The anolyte was 90 mM phosphoric acid and the catholyte was 40 mM NaOH. The ampholyte was a 50/50 mix of a Beckman ampholyte with range of 3-10 and a pharmalyte with a pH range of 8-10.5. Focusing was conducted for 5 min at 10 kV followed by low-pressure mobilization under 10 kV. The protein and standards were detected at 280 nm. Two Bio-Rad (Hercules, Calif.) standards were used for pI calibration, one at pH 10.4 and the other at pH 8.4. The pI of rSEB (9.0+\−0.1) was determined by a two point calibration curve using these two standards.

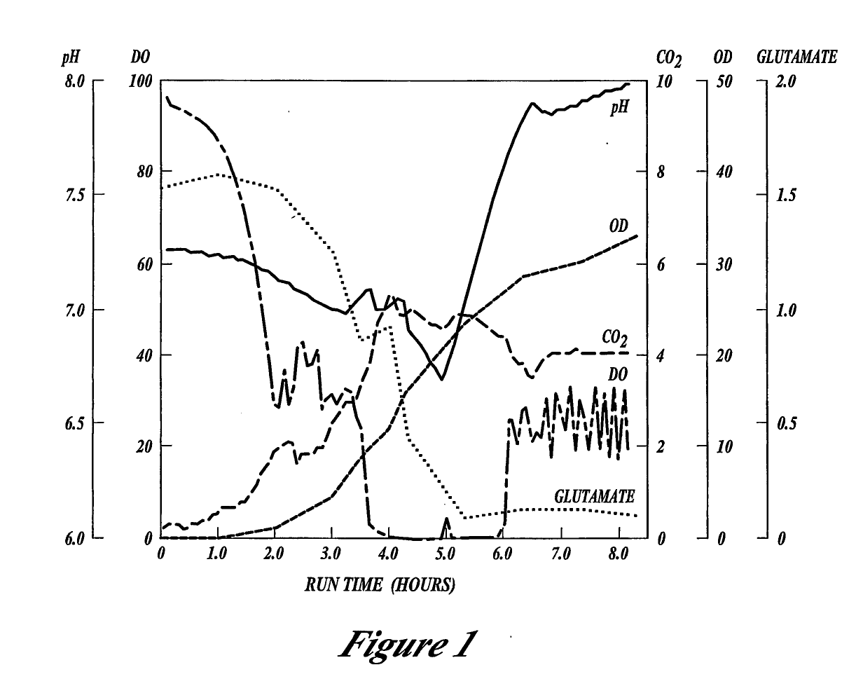

Results from the foregoing Examples demonstrate that the methods and compositions of the invention are generally useful for large-scale fermentation, harvesting, and recovery of rSEB to provide a high yield of clinical grade product for vaccine use. In this regard, FIG. 1 shows the glutamate content, pH, DO₂, percent O₂, agitation rates and OD₆₀₀ values for a representative fermentation at the 80-liter scale. The pH was monitored, but not controlled during the course of the fermentation. The production-scale fermentation was performed in an 80-liter New Brunswick fermenter with an initial working volume of approximately 48 liters. The seed buildup was carried out in two stages. The criteria for the addition of the first 10× nutrient feed was an OD₆₀₀=8±2 or an amount of glutamate in the culture <50% of the starting concentration. When these criteria were met, 2.4 liters of the 10× nutrient stock was aseptically added to the fermenter. Filter-sterilized IPTG was added to a final concentration of 1 mM when the OD₆₀₀ reached 12.5±2.5. The YNB feed solution was added immediately after the addition of the IPTG. Following induction and addition of the YNB feed, the 10× nutrient feed was added to the production culture based on the percent dissolved oxygen (% DO). When the % DO was greater than 30%, the culture was fed at a rate of 50 ml/minute. The nutrient feeding was discontinued when the % DO dropped below 20%. This process was repeated until the fermentation was terminated.

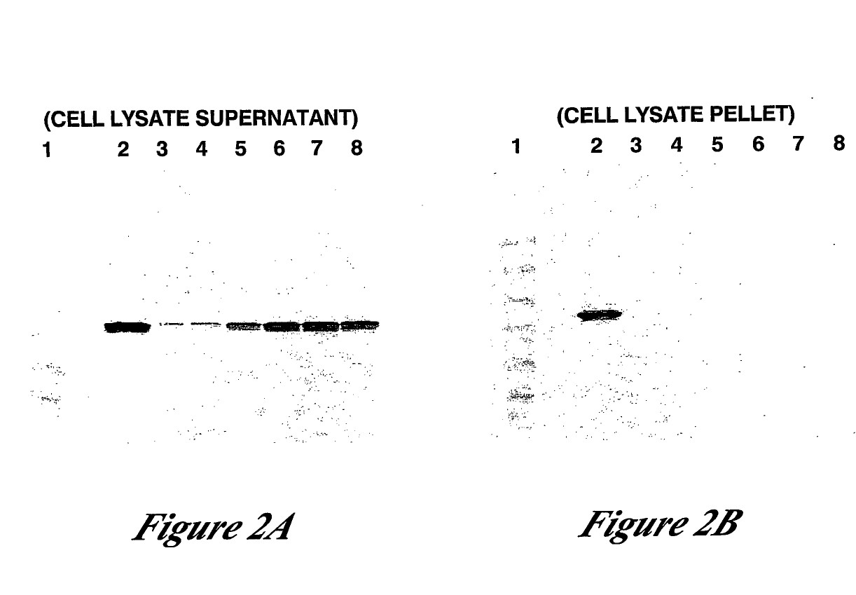

FIGS. 2A and 2B depict the increase in expression of rSEB over the four hours after induction as measured by SDS-PAGE. Under optimum conditions rSEB was observed to be greater than 90% soluble. Prior to fermentation development as described by the present invention, soluble rSEB was limited to less than 50% of the total expressed rSEB, the rest being present in inclusion bodies. Samples were also removed during the induction phase for purity assessment by gram-stain and TSAG plate culture. The harvest was immediately chilled to 10° C., and the agitation was reduced to 55 rpm. End-of-production cells were collected, and the plasmid DNA was isolated and compared to the sequence derived from the MCB. Sequence analysis showed that the nucleotide sequence of the rSEB-coding region conformed to the sequence of the MCB.

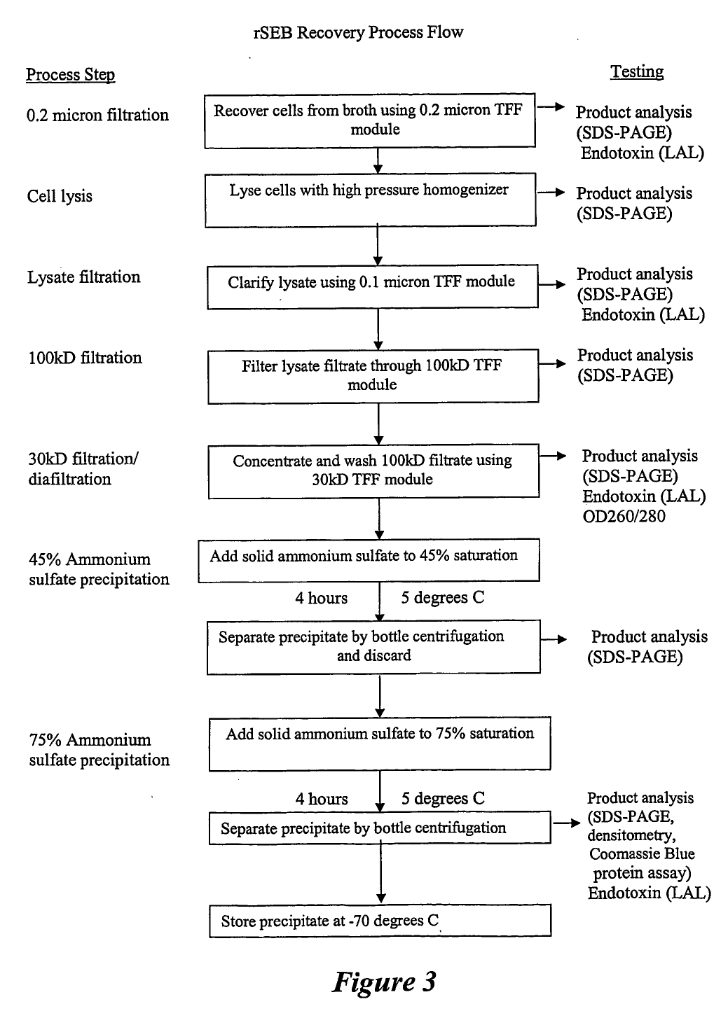

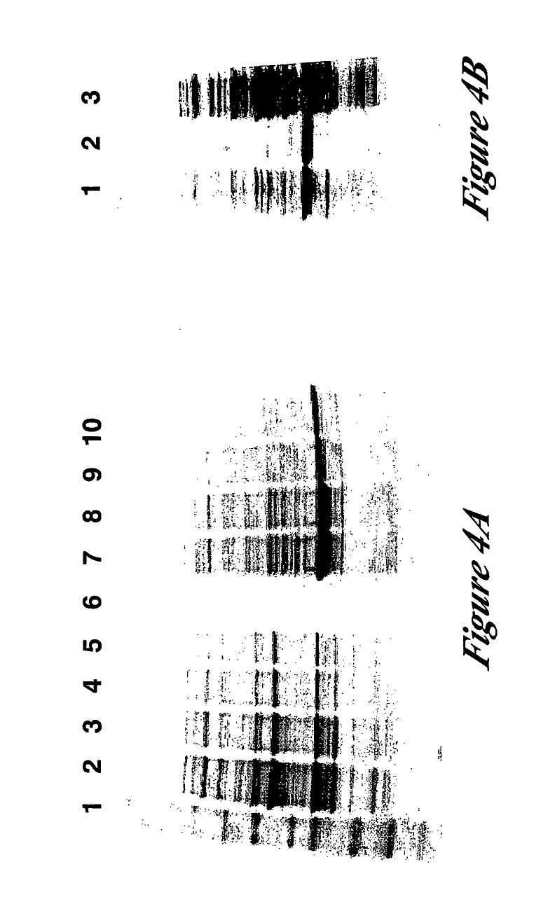

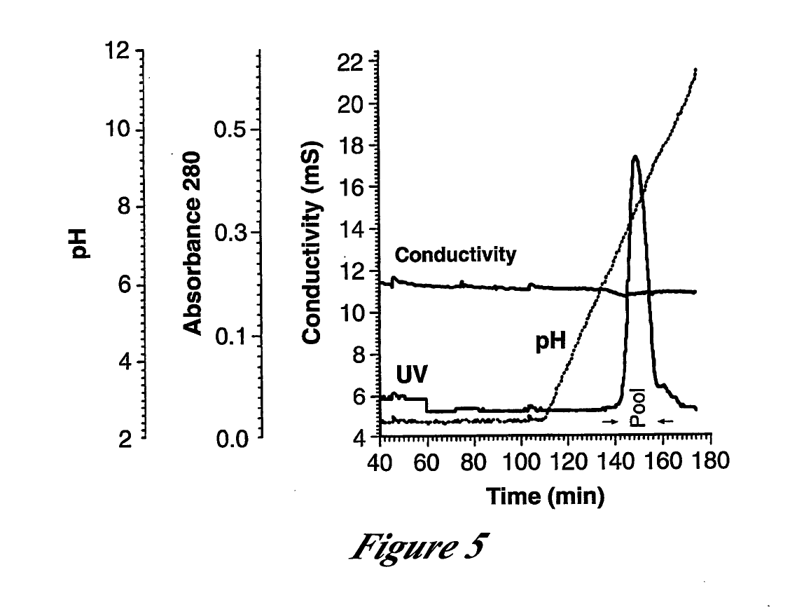

Although proteolysis of the rSEB was not observed by Western blot analysis of in-process samples during the fermentation, it was essential to develop a rapid, high-throughput process for the recovery of the product from the cell paste (Schutte and Kula, Biotechnol. Appl. Biochem. 12:599-620, 1990; Van Reis et al., Biotechnol. Bioeng. 55:737-746, 1997; Bailey and Meagher, Biotechnol. Bioeng. 56:304-310, 1997, each incorporated herein by reference). The large-scale recovery process is shown in FIG. 3. No product degradation was observed during the recovery process as judged by SDS-PAGE and immunoblot analysis. At the end of each of two 80-liter fermentation campaigns, the recovery of crude bulk product was comparable at 247 g and 275 g of ammonium sulfate precipitate. The determination of the protein content by BCA or Pierce Coomassie Plus® assay showed that 10-11% of the precipitate was protein. SDS-PAGE analysis showed the precipitate to be highly enriched (>80%) in rSEB (FIG. 4A). Real-time storage studies over a 12-month period demonstrate that the rSEB remains stable in this form at −70° C. The ability to produce a highly enriched, stable crude bulk form of the vaccine protein was an essential component of designing a high-throughput production system, which was not directly linked to the downstream purification process. This approach allowed for greater flexibility in coordinating unit operations in a multi-product production environment. The average rSEB yield of two fermentations at the 80-liter scale was 350-400 mg of rSEB per liter of culture.

The downstream purification process exemplified herein is summarized in Table 3. Chromatography resin screening and optimization were achieved as described above. Sorbents and buffers were selected that are safe and amenable to the production of a biopharmaceutical for human use (see, e.g., PDA Biotechnology Task Force on Purification and Scale-Up, Industry Perspective on the Validation of Column-Based Separation Processes for the Purification of Proteins, Technical Report No. 14,” J. Parenter. Sci. Technol., 4687-4697, May/June, 1992; Jungbauer and Boschetti, J. Chromatogr. B 662:143-179, 1994, each incorporated herein by reference). The resulting data were then analyzed and utilized in the development of a clinical-scale CGMP process.

TABLE 3
Purification Summary¹

| Sample | Concentration² | Volume (−1) | Total Protein | % Starting | % Previous | EU/ [symbol] |
|---|---|---|---|---|---|---|
| Ammonium | 46.6 | 260 | 12,116 | | | 48 |
| sulfate pellet | | | | | | |
| HIC F/T | 0.86 | 9,450 | 8,127 | 67.08 | 67.28 | < [symbol] |
| G25 #1 | 0.48 | 14,500 | 6,960 | 57.44 | 85.64 | <0 |
| POROS ® 50 HS | 4.01 | 1,401.5 | 5,620 | 46.39 | 80.75 | <0 |
| G25 #2 | 2.31 | 2,288.6 | 5,287 | 43.63 | 107.43 | <0 |
| POROS ® 20 HS | 5.00 | 716 | 3,580 | 29.5 | | |

¹Final yield calculated as 246.73 g precipitate 11.4% protein = 28,127 mg total protein from fermentor; 28,127 mg 29.5% purification yield = 8,297 mg yield rSEB per fermentation; 8,297 mg/56.5L harvest volume = 147 mg/L recoverable yield of rSEB
²Protein content determined by BCA assay expressed in mg/ml.

Considering that the crude bulk product was stable in an ammonium sulfate precipitate, HIC was selected as an initial step in the downstream process. A variety of resins and binding conditions were evaluated for use within the invention. No conditions were identified whereby the product could be desorbed from the resin in sufficiently high yield or without raising concerns about loss of structural integrity. Further investigations were thus undertaken to identify conditions where the rSEB could be recovered in the FT, with the residual contaminants retained on the matrix. In this context, Phenyl-Sepharose Fast-Flow was identified as providing the highest recovery and product purity. Temperature in this step of the recover was identified as a critical factor. SDS-PAGE analysis of the HIC product showed that it was apparently purified to homogeneity (FIG. 4B). Following HIC, LPS levels dropped below the level of detection of the Limulus amoebocyte lysate assay.

The HIC pool was desalted into the POROS® 50HS cation-exhange buffer. Because of the relatively small volume, Sephadex G-25 was used for this purpose. A previously undetected protein contaminant was found to elute at the leading edge of the gradient. N-terminal sequencing identified the contaminant as phosphoglycerate kinase. The product was pooled from fractions free from this or other contaminants detected by SDS-PAGE analysis. Anion exchange chromatography was evaluated as an additional step following POROS® 20S chromatography for ensuring endotoxin and DNA removal. Although there was no detectable impact on yield (rSEB was determined not to bind significantly), there was no apparent gain in purity or activity. At the current scale of production, anion-exchange chromatography was deemed unnecessary but may be valuable at larger production scales to add robustness to the system for the removal of residual endotoxin and DNA.

The product was further refined and concentrated prior to the final polishing/formulation step using POROS® 20HS media (FIG. 5). Small amounts of dimeric and lower molecular weight contaminants were separated form the main product peak. The product from this final step constitutes an intermediate bulk product and has been found to be extremely stable for over 12 months at −70° C.

Because dimerization or the formation of higher-molecular-weight forms of the product could occur during storage, SEC (e.g., using Superdex 75 prep-grade), was selected as the final step in the purification process. The formulation buffer, used as the mobile phase, was selected based upon studies indicating that both structural integrity and adsorption to adjuvant were optimal in this formulation. Recovery from the SEC was approximately 97%.

### EXAMPLE VI

### Purity and Safety Characteristics of rSEB Product

To evaluate the purity and safety characteristics of the rSEB protein produced and purified according to the invention, gram-negative bacterial endotoxin levels were determined by the kinetic Limulus amebocyte lysate method (BioWhittaker, Walkersville, Md.). Endotoxin spike recovery and sample inhibition controls were also run. Total DNA was determined with a DNA Threshold System Molecular Devices, Sunnyvale, Calif.). Negative controls and DNA spike recovery samples were analyzed in parallel. Host cell protein analysis was performed by ELISA using a commercially available kit (Cygnus Technologies, Plainville, Mass.). Residual host cell proteins were detected with a rabbit anti-E. coli antisera and a goat anti-rabbit IgG conjugated to alkaline phosphatase using a kit per the manufacturer's instructions (Cygnus Technologies, Wrentham, Mass.). Negative and positive controls were included. N-terminal sequencing was performed by the Protein Chemistry Laboratory using an ABI 494 CLC protein sequencer equipped with an ABI 785A detector/ABI 140D Microgradient system/ABI 610 data analyzer using GLP protocols developed in their laboratory. Mass Spectrometry was performed by M-Scan (West Chester, Pa.). MALDI-TOF mass spectrometry was performed using a PerSeptive Biosystems Voyager research Station coupled with Delayed Extraction laser-desorption mass spectrometer.

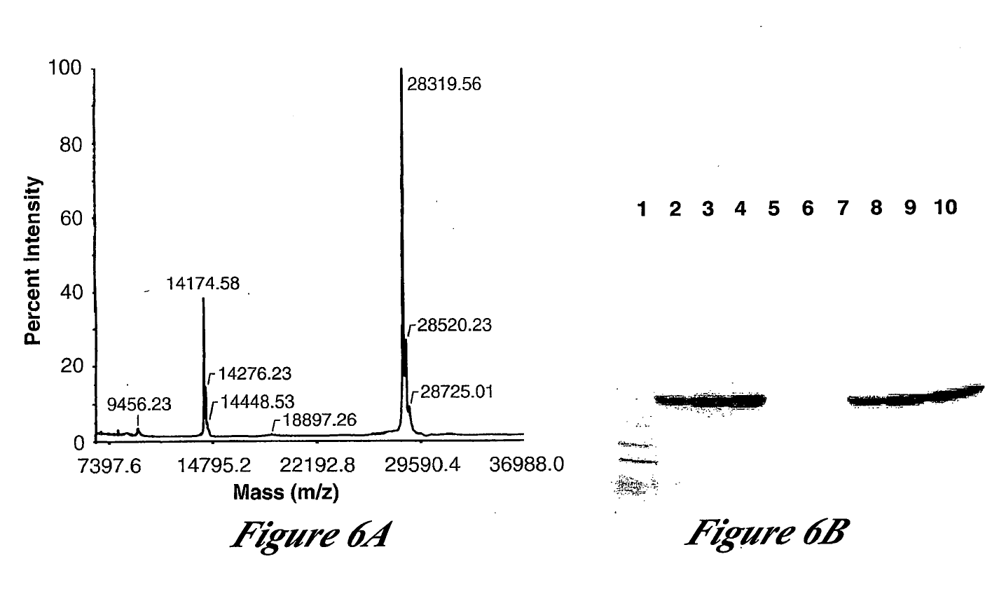

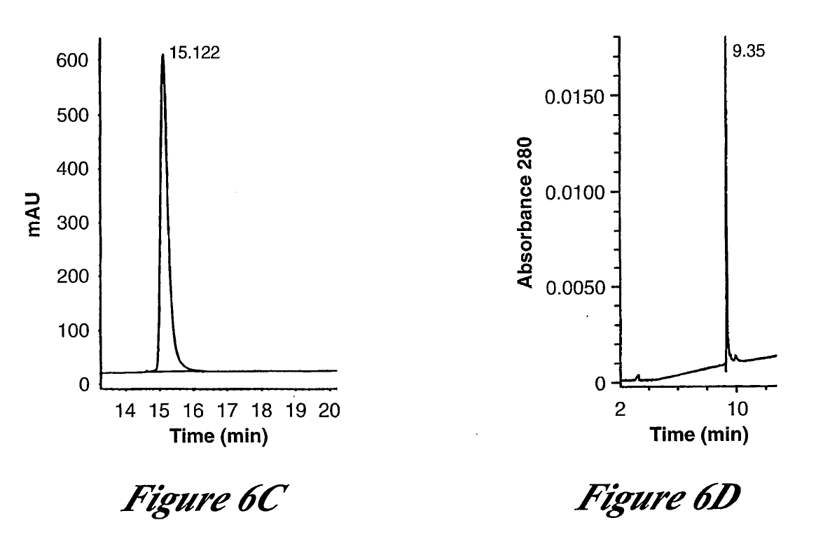

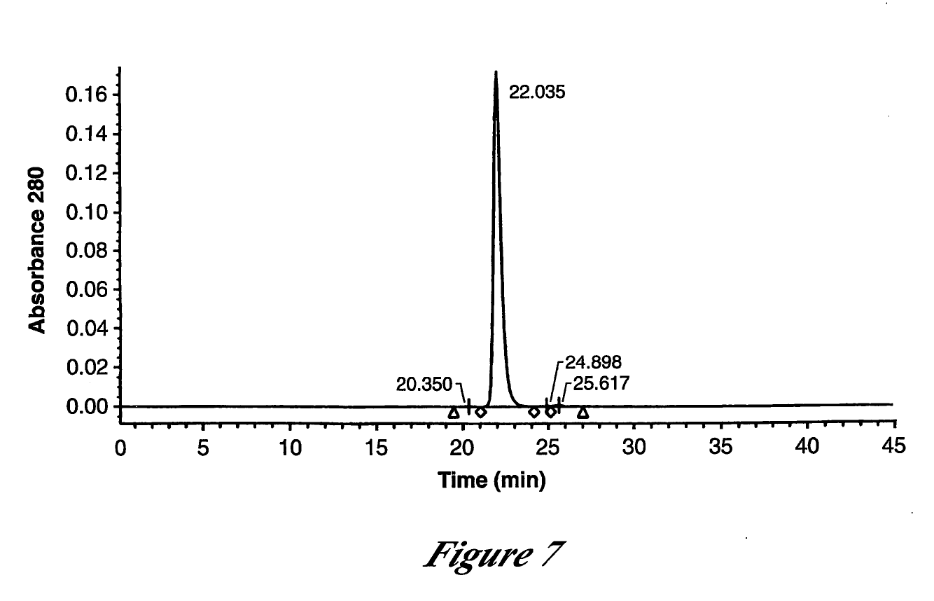

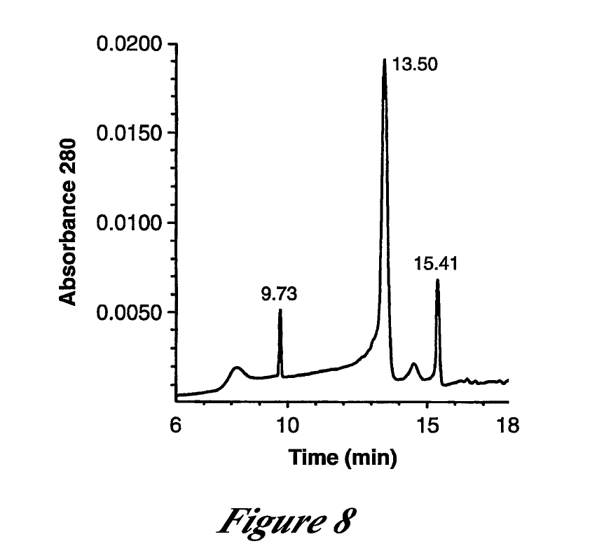

The final purified rSEB product was thus evaluated in extensive detail to determine if it met the physiochemical, safety, and potency attributes required for a Phase I clinical candidate (U.S. Code of Federal Regulations, Title 21, Parts 210, 211 and 600, The National Archives and Records Administration Office of the Federal Register, Government Printing Office, Washington, D.C., 1998). MALDI-TOF analysis showed a single major species with a molecular weight of 28.3 kDa consistent with the predicted size of rSEB. Two higher molecular weight adducts were detected with size increments corresponding to that of the sinnapinic acid matrix (FIG. 6A). Only a single N-terminal amino acid sequence corresponding to the cDNA predicted sequence was identified. SS-PAGE (reduced and non-reduced), reverse phase HPLC on C18 resin, and CZE showed that the protein had been purified to homogeneity FIGS. 6B, 6C, and 6D, respectively). Analytical SEC showed the product to be monomeric in solution (FIG. 6). Capillary IEF showed no evidence of proteolyic degradation or aggregation (FIG. 7). No changes in any of these parameters were observed after approximately 12 months in storage at −70° C.

No E. coli host cell proteins were detected using a validated ELISA assay (LOD=5 ng/mg). Endotoxin and total genomic DNA were below the level of detection (0.06 EU/mg and 15.6 pg/mg, respectively). When tested at a concentration of 1.04 mg/ml, the rSEB final product passed both the USP rabbit pyrogen test and general safety assessment.

### EXAMPLE VII

### Vaccine Efficacy of rSEB

The potency of the rSEB was evaluated in a mouse protection assay (Table 4). Pathogen-free BALB/c mice 10- to 12-weeks old were obtained from Harlan Sprague-Dawley (Frederick Cancer Research and Development Center, Frederick, Md.). Mice were maintained under pathogen-free conditions and fed laboratory chow and water ad libitum. Lipopolysaccharide (LPS) from Escherichia coli O055:B5 was obtained from Difco Laboratories (Detroit) and reconstituted with PBS. Recombinant SEB vaccine was diluted in 0.9% NaCl/50 mM glycine pH 8.5. Mice in groups of 10 were vaccinated intramuscularly with 5 or 20 μg of recombinant SEB vaccine in 100 μl of ALHYDROGEL® adjuvant or the adjuvant alone and boosted at 21 days in the same manner as described for the primary injection. Ten days after the booster vaccination, mice were challenged intraperitoneally with 10 LD₅₀ of wild-type SEB and LPS (75 μg) as described elsewhere (Bavari et al., J. Infect. Dis. 174:33845, 1996, incorporated herein by reference). Three days after challenge, the mice were scored for survivors.

When immunized with 5 μg of rSEB, 70% of the mice challenged with wild-type SEB were protected. 100% of mice were protected when challenged with wild-type SEB following immunization with 20 μg of the recombinant vaccine protein.

TABLE 4
Potency of rSEB Vaccine in a Mouse Protection Assay

| | Dose² | |
|---|---|---|
| Vaccine¹ | (μg/injection) | Live/Total³ |
| Adjuvant | | 0/10 |
| rSEB | 5 | 7/10 |
| rSEB | 20 | 10/10 |

¹The vaccines or adjuvant only were injected intramuscularly at 0 and 21 days.
²The vaccines were adsorbed onto the Alhydrogel adjuvant in 100 μl of glycine-buffered NaCl.
³Mice were challenged with 10 LD₅₀ of wild-type SEB followed 3 h later by a non-lethal, potentiating dose of LPS (75 μg). Survivors were scored three days after the challenge.

Based on the foregoing Examples, the methods and compositions of the invention (exemplified using approximately 106 g of ammonium sulfate pellets (10-11 g rSEB protein), result in an unprecedented high yield recovery of recombinant bacterial SAgs (as evidenced by a final recovery of approximately 3.4 g of final purified rSEB product in the preceding working Examples). The overall recovery of rSEB from ammonium sulfate pelleted protein was approximately 30-34%. This would equate to about 7.9 g of purified and formulated vaccine protein from a single 80-liter fermentation. On this basis, it is clear that the processes and compositions of the invention are robust and highly reproducible. The production of rSEB and other SAgs in large quantities, possessing clinical grade purity, safety and efficacy characteristics, in accordance with the disclosure herein provides important tools for production and use of rSEB vaccines in biodefense and in treatment of diseases and other conditions in mammalian subjects mediated by bacterial SAgs.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation.

What is claimed is:

1. A method for high yield purification of a substantially purified recombinant bacterial superantigen (SAg) suitable for administration to a mammal, comprising the steps of:
contacting a starting load material comprising the recombinant SAg and one or more contaminants to a hydrophobic interaction chromatography (HIC) substrate and washing to HIC substrate;
collecting a flow through fraction from the HIC wash, the flow through fraction comprising HIC-purified recombinant SAg partially or completely separated from the contaminants;
subjecting the HIC-purified recombinant SAg to a suitable buffer exchange to desalt the HIC-purified SAg fractions;
subjecting the HIC-purified recombinant SAg following the buffer exchange to a cation exchange chromatography substrate under conditions sufficient to bind the recombinant SAg to the cation exchange substrate, while not substantially binding the contaminants; and
eluting the recombinant SAg from the cation exchange substrate to provide a high yield substantially purified SAg protein suitable for administration to a mammalian subject.

2. The method of claim 1, wherein the starting load material comprises the recombinant SAg solubilized from an ammonium sulfate precipitation of the recombinant SAg obtained from a recombinant cell lysate.

3. The method of claim 1, wherein the contaminants comprise bacterial endotoxin, DNA or lipopolysaccharide.

4. The method of claim 2, wherein the recombinant SAg following cation exchange is subjected to a second buffer exchange and cation chromatography.

5. The method of claim 2, wherein the recombinant cell lysate is a lysate of recombinant E. coli cells containing an expression construct comprising a recombinant bacterial SAg gene operably linked to one or more expression control elements to direct expression of the recombinant SAg protein upon induction.

6. The method of claim 1, wherein the HIC substrate comprises a propyl, butyl, octyl, or phenyl functional group.

7. The method of claim 6, wherein the HIC substrate comprises a low or high substitution phenyl functional group.

8. The method of claim 7, wherein the HIC resin comprises depyrogenated phenyl-sepharose.

9. The method of claim 1, wherein the cation exchange chromatography separating step follows the HIC step and comprises contacting a mixture comprising an SAg and a contaminant with a cation exchange chromatography resin under conditions in which the recombinant SAg binds to the resin, and eluting the recombinant SAg from the resin under conditions in which the SAg separates from the contaminants.

10. The method of claim 1, wherein the recombinant SAg is recombinant staphylococcal enterotoxin B (rSEB).

11. The method of claim 10, wherein the rSEB is modified by amino acid substitutions at position 89 (from tyrosine to alanine), position 45 (from leucine to arginine), and position 94 (from tyrosine to alanine).

12. The method of claim 1, wherein the substantially purified recombinant SAg comprises at least 90% recombinant SAg.

13. The method of claim 10, wherein the substantially purified recombinant staphylococcal enterotoxin B comprises at least 90% rSEB.

14. The method of claim 1, wherein the substantially purified recombinant SAg is essentially free of one or more contaminants comprising bacterial endotoxin, DNA or lipopolysaccharide.

15. A recombinant bacterial superantigen (rSAg) composition produced according to the method of claim 1.

16. A bacterial fermentation process for high yield production of a recombinant bacterial superantigen (SAg), comprising the steps of:
culturing E. coli cells of a Master Cell Bank containing an expression construct comprising a recombinant bacterial SAg gene operably linked to one or more expression control elements to direct expression of a recombinant SAg protein following induction in a sterile seed medium to yield a seed culture;
culturing the recombinant E. coli cells from the seed culture in a sterile production medium to yield a production culture;
inducing the recombinant E. coli cells of the production culture to express the recombinant SAg protein;
disrupting the recombinant E. coli cells from said production culture to yield a lysate containing the recombinant SAg protein; and
recovering the recombinant SAg from the lysate, wherein at least about 50-60% of the recombinant SAg is recovered in a soluble form.

17. The method of claim 16, wherein greater than 60% of the recombinant SAg is recovered in a soluble form.

18. The method of claim 16, wherein at least 75-80% of the recombinant SAg is recovered in a soluble form.

19. The method of claim 16, wherein about 90% or more of said bacterial SAg is recovered in a soluble form.

20. The method of claim 16, comprising culturing the recombinant E. coli cells of the seed culture in sterile seed medium to form a second seed culture; and culturing the recombinant E. coli cells of the second seed culture in sterile production medium to from the production culture.

21. The method of claim 16, wherein the lysate is substantially free of SAg in an aggregate form as determined by the presence of inclusion bodies.

22. The method of claim 16, wherein the recombinant SAg is a recombinant staphylococcal enterotoxin (SE).

23. The method of claim 22, wherein the recombinant SE is a recombinant SEB (rSEB).

24. The method of claim 22, wherein the rSEB is modified by acid substitutions at position 89 (from tyrosine to alanine), position 45 (from leucine to arginine), and position 94 (from tyrosine to alanine).

25. The method of claim 16, wherein the seed medium is a yeast extract-based culture medium.

26. The method of claim 16, wherein the seed medium comprises one or more of a trace element comprising NH₄SO₄, ZnSO₄.7H₂O, CuSO₄.5H₂O, MnSO₄.H₂O, FeCl₃.6H₂O, CoCl₂.6H₂O, or Na₂MoO₄.2H₂O.

27. The method of claim 16, wherein the seed medium excludes added glucose.

28. The method of claim 16, wherein the seed medium excludes animal nitrogen sources.

29. The method of claim 16, wherein the seed medium comprises tryptone or soytone as a non-animal nitrogen source.

30. The method of claim 16, wherein induction of the production culture is performed when the production culture exhibits an OD₆₀₀ of about 5 to about 20.

31. The method of claim 30, wherein induction of the production culture is performed when the production culture exhibits an OD₆₀₀ of about 10 to about 15.
